(19) United States
(12) Patent Application Publication
(10) Pub. No.: US 2018/0112248 A1
(43) Pub. Date: Apr. 26, 2018

(54) FLOW CELL APPARATUS AND METHOD OF ANALYSING BIOFILM DEVELOPMENT

(71) Applicants: Nanyang Technological University, Singapore (SG); National University of Singapore, Singapore (SG)

(72) Inventors: Yee Cheong Lam, Singapore (SG); Chun Ping Lim, Singapore (SG); Quoc Mai Phuong Nguyen, Singapore (SG); Yehuda Cohen, Singapore (SG)

(21) Appl. No.: 15/560,846

(22) PCT Filed: Mar. 22, 2016
(86) PCT No.: PCT/SG2016/050131

§ 371 Date: Sep. 22, 2017

(30) Foreign Application Priority Data
Mar. 23, 2015 (SG) 10201502251P

(51) Int. Cl. C12Q 1/02; B01L 3/00

(52) U.S. Cl. CPC C12Q 1/02; B01L 3/502715; B01L 3/502746; B01L 3/502738; B01L 2300/0861; B01L 2300/0877; B01L 2200/0689; B01L 2200/025; B01L 2300/0887

(57) ABSTRACT

A flow cell apparatus including a channel plate having a channel recessed into a surface of the channel plate, and a groove recessed into the surface of the channel plate, the groove configured to surround the channel and preferably along a boundary of the channel. The flow cell apparatus further includes a seal shaped and receivable in the groove, a substrate, a backing plate, and a fastening element configured to removably attach the channel plate to the backing plate with the substrate sandwiched between the channel plate and the backing plate to bear the seal against the channel plate with the substrate.

### CROSS-REFERENCE TO RELATED APPLICATIONS

The present application claims the benefit of the Singapore patent application No. 10201502251P filed on 23 Mar. 2015, the entire contents of which are incorporated herein by reference for all purposes.

### TECHNICAL FIELD

Embodiments relate generally to flow cell apparatus and method of analysing biofilm development.

### BACKGROUND

Biofilms are aggregates of microorganisms on surfaces/interfaces and are bound by an extra-cellular polymeric matrix. Biofilms form on almost every surface/interface in very diverse environments. The aggregation of bacteria in close physical contact allows for fast diffusion of a variety of small signaling molecules to serve as interspecies signals resulting in many different behavioral patterns compared to those observed by the individual members of these communities. Biofilms are inherently heterogeneous and their development is closely correlated to environmental gradients, both in the environment that they reside and within the biofilms themselves. These communities of microorganisms are very resilient and can adapt to environmental changes. Hence, robust environmental control is required to study such a heterogeneous and dynamic system under well-defined conditions. The study of biofilms using conventional microbiology techniques such as agar plates only enabled biofilm growth to be investigated in static environments and without a rigorous control over the environments. However, the variation of their immediate environments will have significant effects on the behavior of biofilms.

The study of biofilms in flow cells is important as biofilms rarely grow in static environments in nature. Conventional techniques such as Petri dish and microtiter plate are unable to generate reproducible dynamic environments for biofilm studies. Commonly used flow cells typically include straight channels with growth media pumped by peristaltic pumps. The protocols for using such flow cells were well documented. Besides uni-directional flow field, flow cells that can generate two-dimensional flow fields had also been developed. These flow cells offer simple platforms for research on biofilms but are limited in their ability to generate specific well-defined micro-scale conditions in the flow cells.

The convergence of microfluidics and biofilm flow cells has resulted in flow cells that can create environments with defined factors such as hydrodynamic stresses, chemical gradients and temperature gradients. Nonetheless, the developed micro-channels are often custom-designed for specific experiments and the combinatorial gradients are usually not controllable. Furthermore, the substrate is restricted by the fabrication technique of microfluidic channels and the microfluidic channels are usually sealed permanently.

Example embodiments provide flow cell apparatus and method of analysing biofilm development that seek to address at least some of the issues identified above.

### SUMMARY

According to various embodiments, there is provided a flow cell apparatus including a channel plate having a channel recessed into a surface of the channel plate, and a groove recessed into the same surface of the channel plate, with the groove configured to surround the channel and preferably along a boundary of the channel; a seal shaped and receivable in the groove; a substrate; a backing plate; and a fastening element configured to removably attach the channel plate to the backing plate with the substrate sandwiched between the channel plate and the backing plate to bear the seal against the channel plate with the substrate.

According to various embodiments, there is provided a channel plate including a channel recessed into a surface of the channel plate; and a groove recessed into the surface of the channel plate, with the groove configured to surround the channel and preferably along a boundary of the channel, wherein the channel plate is configured to be removably attachable to a backing plate by a fastening element with a substrate sandwiched between the channel plate and the backing plate to bear a seal received in the groove against the channel plate with the substrate.

According to various embodiments, there is provided a method of analysing biofilm development, the method comprising quantifying biofilm development in the flow cell apparatus as described herein or in the channel plate as described herein based on bio-volume.

According to various embodiments, there is provided a flow system including a flow cell apparatus as described herein, a valve connected to the channel of the flow cell apparatus, and a collector connected to the valve. The valve may be a three or more way valve. The valve may be configured to direct a fluid flow through the valve into the channel of the flow cell apparatus in a flow mode, and further configured to direct the fluid flow through the valve into the collector and hold the fluid in the channel of the flow cell apparatus in a locked mode.

According to various embodiments, there is provided a flow system including a channel plate as described herein, a valve connected to the channel of the channel plate, and a collector connected to the valve. The valve may be configured to direct a fluid flow through the valve into the channel of the channel plate in a flow mode, and further configured to direct the fluid flow through the valve into the collector and hold the fluid in the channel of the channel plate in a locked mode.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1A shows a schematic block diagram of a flow apparatus according to various embodiments;

FIGS. 1B and 1C show schematic diagrams illustrating methods of analysing biofilm developments according to various embodiments;

FIGS. 2A and 2B show channels which may generate a controllable, well-defined and reproducible environment for biofilm studies according to various embodiments;

FIGS. 3A and 3B show the controllable velocity field in the channel of FIG. 2A in which a hyperbolic expansion may be used according to various embodiments;

FIGS. 4A and 4B show the controllable concentration in the channel of FIG. 2A in which a hyperbolic expansion may be used according to various embodiments;

FIGS. 5A and 5B show an assembled view and an exploded view of a flow cell apparatus according to various embodiments;

FIG. 5C shows a channel plate of the flow cell apparatus of FIGS. 5A and 5B according to various embodiments;

FIG. 5D shows a backing plate of the flow cell apparatus of FIGS. 5A and 5B according to various embodiments;

FIG. 5E shows a backing plate according to various embodiments;

FIG. 5F shows a channel plate according to various embodiments;

FIGS. 5G and 5H show an exploded view and an assembled view of a channel plate according to various embodiments;

FIGS. 6A, 6B and 6C show an assembled view, an exploded view and a side view of a flow cell apparatus according to various embodiments;

FIG. 7A shows a schematic diagram of assembling the flow cell apparatus of FIGS. 6A to 6C with the aid of a fixture according to various embodiments;

FIG. 7B shows the fixture used in FIG. 7A according to various embodiments;

FIG. 8 shows a schematic diagram of dismantling the flow cell apparatus of FIGS. 6A to 6C according to various embodiments;

FIGS. 9A to 9C show confocal microscopy images of monospecies biofilm formed after 3.5 hours growth at room temperature (25° C.) at various locations along the hyperbolic expansion of the channel of FIG. 3A according to various embodiments;

FIGS. 10A to 10C show confocal microscopy images of a 3-day-old multispecies biofilm cultured at room temperature (25° C.) at different positions along the hyperbolic expansion of the channel of FIG. 3A according to various embodiments;

FIG. 11A shows the top view of a channel with a hyperbolic channel profile according to various embodiments;

FIG. 11B shows a set-up of a flow cell system according to various embodiments;

FIG. 11C shows a graph illustrating the comparison of simulated (continuous line) and measured (dashed line) flow velocity of a channel according to various embodiments;

FIG. 11D shows a schematic diagram of simulated mid-plane flow field according to various embodiments;

FIG. 12 shows a flow diagram of an experimental procedure of biofilm growth experiment according to various embodiments;

FIGS. 13A and 13B show schematic diagrams of a flow cell system set-up in two different modes of operation according to various embodiments;

FIGS. 14A to 14D show experiment data illustrating the dynamic nature of P. putida OUS82::GFP biofilm formation and dispersal at low flow rate Q=0.1 ml h⁻¹ per inlet according to various embodiments;

FIGS. 15A to 15D show experimental data illustrating the dynamics of P. putida OUS82::GFP clusters formation and dispersal at position 7a at significant time-points under low flow rate Q=0.1 ml h⁻¹ per inlet according to various embodiments;

FIGS. 16A and 16B show microcolonies structure of P. putida OUS82::GFP model biofilm developed at position 7a under low flow according to various embodiments;

FIG. 17 shows a graph illustrating the doubling time of biofilm at position 7a over defined periods of the experiment according to various embodiments.

### DETAILED DESCRIPTION

Embodiments described below in context of the apparatus are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural referents unless context clearly indicates otherwise. Similarly, the word “or” is intended to include “and” unless the context clearly indicates otherwise.

According to various embodiments, there is provided a flow cell apparatus configured to generate controllable, well-defined and reproducible environment for both monospecies and multispecies biofilm research. The flow cell apparatus may facilitate biofilm studies such as, but not exclusively confined to, microorganism attachment, biofilm development and dispersal in a well-controlled, reproducible and/or changing environment. The environments generated may include physical and chemical factors. Physical parameters may include, but not restricted to, hydrodynamic stresses and temperature. Chemical factors may include, but not limited to, chemical concentration, gas concentration, surface energy and wettability of the substrate and the channel walls. Biofilm dynamics in response to steady environments and to changes in the environments may also be elucidated by employing the flow cell apparatus. The flow cell apparatus may have a minimum of one inlet, but generally at least two inlets, for feeding into a channel leading into one outlet or more with one outlet being generally preferred. The channel profile may be varied for the generation of various physical and chemical environments, such as but not limited to, shear rate gradient and chemical gradient by using an expanded region followed by a contracted region. Using a hyperbolic expansion as the expanded region, a linear shear rate gradient in the center region of the channel may be generated. In conventional microfluidics and biofilm flow cells, controlling shear gradient as a factor is unheard of. By infusing fluids and/or gas of different concentrations into the two inlets respectively, a well-defined chemical gradient across the width of the channel may also be generated. In addition, the chemical gradient at the centerline of the expanded region may decrease along the channel giving rise to the second order derivative of chemical concentration.

The flow cell apparatus may have a characteristic dimension in the order of 100 μm but not limited to it, and may operate in laminar flow regime such that fluidic conditions in the channel may be controllable, well-defined and reproducible. The flow cell apparatus may also enable the control of the environment with micrometer resolution to generate physical and chemical micro-environments. The well-defined environments in the flow cell apparatus may be predicted by fluid dynamics simulation and verified experimentally.

In addition, the flow cell apparatus may have a removable surface (hereinafter referred to as the substrate) for microorganism attachment and biofilm growth. The substrate may be, but not limited to, a microscope coverslip or a polymer sheet/film. As the substrate is removable, it may be subjected to pre-treatment of its surface and the biofilm developed on the substrate may be subjected to post-analysis. Pre-treatment of the substrate may be, but not exclusive to, surface modification or surface patterning. The substrate may also be removed at any point of the experiment, with the biofilm intact, for downstream analysis such as, but not limited to, meta-omics analyses, atomic force microscopy or scanning electron microscopy.

The substrate and/or the channel may have one or more markers on the surfaces, with minimum three markers being generally preferred, for using as spatial reference points such as, but not limited to, during assembly, flow cell operation and post-analysis of biofilm on the substrate. The markers may be fabricated by, but not exclusive to, laser marking, etching or machining.

The flow cell apparatus may include a channel plate containing channel features and a sealing seat, a gasket, the removable substrate, a primary backing plate that contains a window for observations and the seat for the removable substrate for alignment of the substrate and fast assembly of the flow cell apparatus, with or without at least one additional backing plate, and fasteners. The flow cell may be sealed using a gasket with a sealing seat, with an O-ring seated in an O-ring groove following the profile of the channel features being particularly preferred, that is sandwiched between the channel plate and at least one or more backing plates. The channel plate may be disposable and the backing plate(s) may be reusable. The channel features on the channel plate may have arbitrary two-dimensional and/or three-dimensional profile such as, but not limited to, a hyperbolic expansion.

The channel plate of the flow cell apparatus may contain one or more channels. Each of the channels may have their independent channel features. Furthermore, each of the channels may have their independent seals using a gasket with a sealing seat, with O-rings seated in O-ring grooves being particularly preferred. In addition, the inflows and outflows of each channel may be independent. The environments in the channels, which may include, but not limited to, flow fields and chemical conditions, may be independently controlled. Therefore, multiplexed experiments may be conducted simultaneously on a single flow cell apparatus.

The channel plate may include two or more layers or panels with channel features. The layers may be attached together by, but not limited to, bonding techniques such as thermal bonding, laser bonding, etc. The combination of features on different layers may enable the formation of three-dimensional features in the channel.

The backing plates may enhance the sealing by increasing the rigidity of the assembly and ensuring the flatness of the sealed flow cell. The rim of the observation window on the primary backing plate may be tapered to improve the accessibility of observation or measurement tools to regions of interest on the substrate. A winged backing plate, i.e. a backing plate with one or more wings added to its sides, with two wings generally being preferred, may be used as an alternative to provide firm clamping of the device during measurement and analysis such as fixing on microscope sample holder. The required sealing force may be provided by fasteners such as, but not exclusive to, bolts and nuts, snapfit fasteners together with compression springs, or clamps.

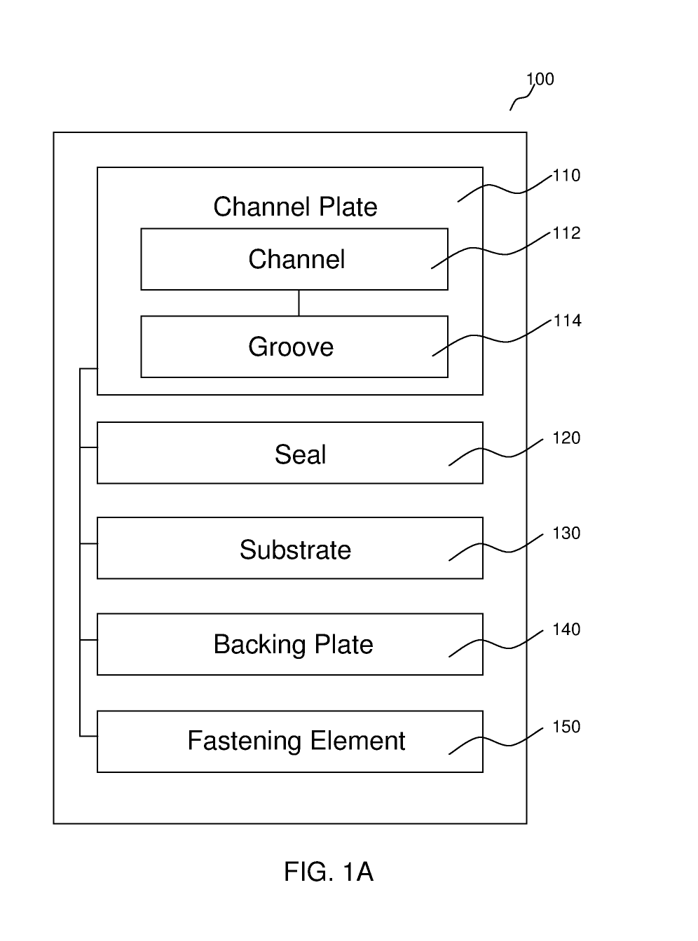

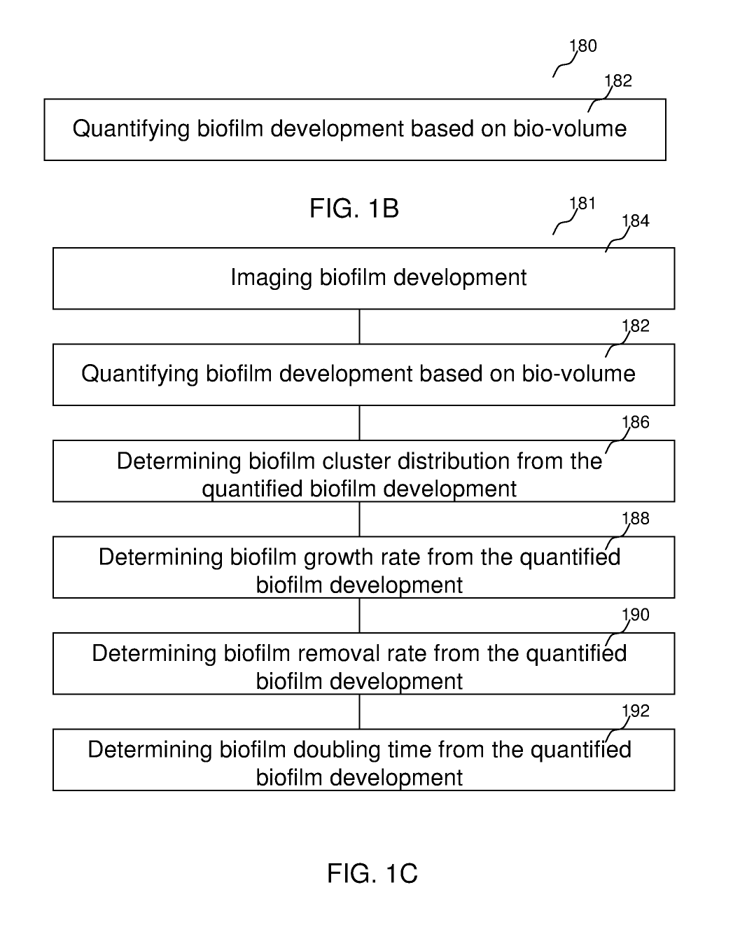

FIG. 1A shows a schematic diagram of a flow cell apparatus 100 according to various embodiments. The flow cell apparatus 100 may include a channel plate 110. The channel plate 110 may be in the form of a panel or a layer. The channel plate 110 may include a channel 112 recessed into a surface of the channel plate 110. The channel 112 may be in the form of a trench cut into the surface of the channel plate 110. The channel plate 110 may further include a groove 114 recessed into the surface of the channel plate 110. The groove 114 may be in the form of an indentation or a notch on the surface of the channel plate 110. The groove 114 may be configured to surround the channel 112. The groove 114 may be configured to surround the channel 112 along a boundary of the channel 112. Accordingly, the groove 114 may be formed to enclose the channel 112 on the surface of the channel plate 110 and to follow the shape or profile of the channel 112. The flow cell apparatus 100 may further include a seal 120 shaped and receivable in the groove 114. The seal 120 may be in the form of a gasket or an O-ring. The seal 120 may also have a shape which corresponds to the profile of the groove 114 such that the seal 120 may be inserted into the groove 114. The flow cell apparatus 100 may further include a substrate 130, a backing plate 140, and a fastening element 150. The substrate 130 may include a surface suitable for microorganism attachment and biofilm growth, for example a microscope coverslip or a polymer sheet/film. The backing plate 140 may be in the form of a panel or a layer. The fastening element 150 may be configured to removably attach the channel plate 110 to the backing plate 140 with the substrate 130 sandwiched between the channel plate 110 and the backing plate 140 to bear the seal 120 against the channel plate 110 with the substrate 130. Accordingly, the fastening element 150 may be in direct contact with the channel plate 110 and the backing plate 140 to hold the channel plate 110 and the backing plate 140 together such that the substrate 130 may be between the channel plate 110 and the backing plate 140. In this manner, the substrate 130 may hold the seal 120 against the channel plate 110 such that the channel 112 may be sealed and leak-proof. The fastening element 150 may further allow the channel plate 110 and the backing plate 140 to be detached such that the substrate 130 may be removed.

In other words, the flow cell apparatus 100 may include a channel component having a flow canal formed into a surface of the channel component. The channel component may further include a long narrow indentation on the surface of the channel component, the indentation may be configured to follow the shape of the flow canal. The flow cell apparatus 100 may further include a pliable material configured to be insertable into the indentation. The flow cell apparatus 100 may further include a sheet or a film with a surface suitable for biofilm growth. The flow cell apparatus 100 may further include a support component and a securing component. The support component may be configured to be attached to the channel component such that the sheet may be between the support component and the channel component. Further, the pliable material may be held between the sheet and the channel component such that the pliable material may be inserted into the indentation forming an air-tight seal around the flow canal. The securing component may be configured to attach the support component to the channel component. The securing component may further be configured to allow the support component and the channel component to be separated such that the sheet may be removed from the flow cell apparatus 100.

According to various embodiments, the channel 112 may be configured to define a desired flow environment. The desired flow environment may be a controllable, well-defined and reproducible environment for biofilm studies. The desired flow environment may include, but not limited to, a desired physical and/or chemical condition. Physical conditions may include hydrodynamic stress, or temperature, or shear rate gradient, etc. Chemical conditions may include chemical concentration, or gas concentration, or surface energy, or wettability of the substrate 130 and the channel wall, etc. The channel 112 may be shaped or profiled such that the channel 112 may establish the physical and/or chemical conditions for the desired flow environment. The channel 112 may include a two-dimensional or a three-dimensional channel profile.

According to various embodiments, the desired flow environment may include a predetermined shear rate gradient along the channel 112. For example, a decreasing shear rate gradient may be established along the channel 112 by shaping the channel 112 to adopt a hyperbolic expansion profile. An increasing shear rate gradient may be established along the channel 112 by shaping the channel 112 to adopt the hyperbolic contraction profile.

According to various embodiments, the channel 112 may include an expanded region followed by a contracted region. The expanded region may include a hyperbolic expanded region.

According to various embodiments, the channel 112 may include a contracted channel. The contracted channel may include a hyperbolic contracted channel.

According to various embodiments, the channel plate 110 may further include an inlet at an end of the channel 112 and an outlet at another end of the channel 112. Accordingly, the channel plate 110 may have at least one inlet in fluid communication with the first end of the channel 112 and at least one outlet in fluid communication with the second end of the channel 112. When the channel 112 has two or more inlets, a combination of different fluids and flow rates may be infused into the respective inlets to control the flow environment in the channel 112. When the channel 112 has one outlet, all the fluids infused into the channel 112 may converge into the one outlet. According to an implementation, the channel 112 may have two inlets and one outlet.

According to various embodiments, the backing plate 140 may include a window. The window may be an opening in the backing plate 140 such that when the channel plate 110 and the backing plate 140 are attached together to sandwich the substrate 130, the substrate 130 may be observed through the window in the backing plate 140.

According to various embodiments, an edge of the window in the backing plate 140 may be tapered. Accordingly, at the edge or the rim of the window, the backing plate 140 may become thinner towards the opening forming the window. The tapered edge of the window may facilitate observation or measurement tools to be deployed in the vicinity of the window for capturing observations or measurements in specific locations of the substrate 130 within the window.

According to various embodiments, the backing plate 140 may include a recessed portion for receiving the substrate 130. The recessed portion may be formed along the edges of the window. The recessed portion may function as a seat or a holder for receiving and aligning the substrate 130 into the backing plate 140 such that the substrate 130 may be observed through the window in the backing plate 140.

According to various embodiments, the backing plate 140 may further include a winged portion. The winged portion may be an extension portion or a flange extending from a side of the backing plate 140. The winged portion may facilitate fixing the flow cell apparatus 100 to an observation or measurement tool, such as a microscope etc. For example, the winged portion may facilitate clamping of the backing plate 140 of the flow cell apparatus 100 to a holder of the microscope. According to various embodiments, the backing plate 140 may include one or more winged portion extending from its sides. According to an implementation, the backing plate 140 may include two winged portion extending from its side.

According to various embodiments, the fastening element 150 may be configured to provide sufficient force to attach the channel plate 110 to the backing plate 140 with the substrate 130 sandwiched between the channel plate 110 and the backing plate 140, and for the seal 120 to be sandwiched between the channel plate 110 and the substrate 130. The fastening element may further be configured to allow the channel plate 110 to be detached from the backing plate 140 such that the substrate 130 may be removed.

According to various embodiments, the flow cell apparatus 100 may include two or more fastening elements 150. The number of the fastening elements 150 may vary. According to an implementation, there may be four fastening elements 150. According to various embodiments, the two or more fastening elements 150 may be configured to self-balance and evenly distribute a compressive stress applied to attach the channel plate 110 to the backing plate 140. Accordingly, the two or more fastening elements 150 may be arranged such that the compressive stress applied by the fastening elements 150 to attach the channel plate 110 to the backing plate 140 may be balanced and evenly distributed across the channel plate 110 and/or the backing plate 140.

According to various embodiments, the fastening element 150 may be configured to compensate for thickness variation of the channel plate 110 and/or the backing plate 140. Variation in the plate's thickness may be due to but not limited to the fabrication process. Accordingly, when attaching the channel plate 110 to the backing plate 140 with the fastening element 150, the fastening element 150 may compensate the variation of the channel plate 110 and/or the backing plate 140 such that the quality of the sealing between the channel plate 110 and the backing plate 140 may not be affected by the variation in thickness of the plates, and sealing quality may be maintained.

According to various embodiments, the fastening element 150 may be configured to self-lock. Accordingly, the fastening element 150 may lock the channel plate 110 and the backing plate 140 together upon attaching the fastening element 150 to the channel plate 110 and the backing plate 140. Thus, the channel plate 110 and the backing plate 140 may be easily assembled together with the fastening element 150.

According to various embodiments, the fastening element 150 may include a quick-release fastening element. Accordingly, the fastening element 150 may be released quickly for separating the channel plate 110 from the backing plate 140. Thus, the fastening element 150 may be easily released to disassemble the channel plate 110 and the backing plate 140.

According to various embodiments, the fastening element 150 may include a snapfit fastener and a compression spring. In this configuration, the channel plate 110 and the backing plate 140 may include a through hole respectively. The snapfit fastener may first be passed through the compression spring in an axial direction such that an end of the compression spring may rest on the head of the snapfit fastener. The snapfit fastener may then be passed through the hole in the channel plate 110 and the hole in the backing plate 140 such that another end of the compression spring may bear against a surface of the channel plate 110. A catch or a locking feature at the tip of the snapfit fastener may hook on a surface of the backing plate 140 after the tip of the snapfit fastener has passed through the hole in the backing plate 140. In this manner, the compression spring and the catch at the tip of the snapfit fastener may hold the channel plate 110 and the backing plate 140 together. Accordingly, the snapfit fastener and the compression spring may allow a self-locking assembly of the flow cell apparatus 100. Further, the snapfit fasteners and the compression springs may provide the necessary sealing force or compressive stress to attach the channel plate 110 to the backing plate 140. The compressive stress applied may be self-balanced by the resistance of the compression springs as the compression springs are compressed and constrained by the snapfit fasteners' heads and the channel plate 110. In addition, any variation in the plates' thicknesses due to fabrication process may be compensated and thus may not have any effects on the sealing quality as the sealing force required is self-adjusted by the compression springs. According to various embodiments, the snapfit fastener and the compression spring may easily assemble the channel plate 110 to the backing plate 140 by pressing and releasing the snapfit fastener head. The snapfit fastener and the compression spring may also be easily released to separate the channel plate 110 and the backing plate 140 by pressing the snapfit fastener's head and cutting the catch or the locking feature.

According to various embodiments, assembling the flow cell apparatus 100 may be aided by a fixture. The fixture may include a box shaped body with a through hole in the centre. The through hole may include a ledge such that the backing plate 140 and the channel plate 110 may be placed in the through hole and rested on the ledge. In this manner, the fixture may allow a quick assembly of the flow cell apparatus via a fast “press-and-release” of the snapfit fastener with the compression spring into the channel plate 110 and the backing plate 140.

According to various embodiments, the fastening element 150 may include a bolt and a nut. In this configuration, the channel plate 110 and the backing plate 140 may include a through hole respectively. The bolt may be passed through the hole in the channel plate 110 and the hole in the backing plate 140 such that the bolt head is rested on a surface of the channel plate. The nut may then be screwed onto the threaded shaft of the bolt from an end of the bolt opposite the bolt head until the nut bears against a surface of the backing plate 140 such that the channel plate 110 and the backing plate 140 may be held together by the bolt and nut configuration. The bolt and nut configuration may provide the necessary sealing force or compressive stress to attach the channel plate 110 to the backing plate 140. The compressive stress applied may be evenly distributed by evenly tightening two or more sets of the bolt and nut holding the channel plate 110 and the backing plate 140 together. In addition, any variation in the plates' thicknesses due to fabrication process may be compensated by manual adjustment of the bolt and nut, and thus may not have any effects on the sealing quality.

According to various embodiments, the fastening element 150 may include a clamp. The clamp may be a C-clamp. The C-clamp may clamp the channel plate 110 and the backing plate 140 together.

According to various embodiments, the substrate 130 may include one or more markers. According to various embodiments, the channel plate 110 may include one or more markers. The markers may be used as spatial reference points during assembling of the flow cell apparatus, conducting of the flow cell operation and/or post-analysis of biofilm developed on the substrate. According to various embodiments, the substrate 130 or the channel plate 110 may include at least three markers.

According to various embodiments, the channel plate 110 may include two or more channels 112 recessed into the surface of the channel plate 110. With two or more separate channels 112 formed in the channel plate 110, the flow cell apparatus may be used for conducting two or more flow analysis concurrently. The flow inlets (or inflows) and flow outlets (or outflows) of each of the two or more channels 112 may be independent of each other. The desired flow environment in each of the two or more channels 112 may be different from each other and may be independently controllable. The flow environment may include, but not limited to, flow fields and chemical conditions. Accordingly, multiplexed experiments may be conducted simultaneously on a single flow cell apparatus.

According to various embodiments, each channel of the two or more channels 112 may include a channel profile different from each other. Accordingly, flow analysis for different channel profiles may be conducted concurrently. According to various embodiments, a channel profile of each of the two or more channels 112 may be the same.

According to various embodiments, the channel plate 110 may include two or more grooves 114. Each of the two or more grooves may be configured to surround a corresponding channel of the two or more channels 112 along the boundary of the corresponding channel. Accordingly, each of the two or more channels 112 may be sealed separately by different seals 120. Thus, the seal 120 of the flow cell apparatus 100 may include two or more separate and independent seals, such as gaskets or O-rings.

According to various embodiments, the channel plate 110 may include two or more layers. A first layer of the two or more layers may include a base channel recessed into a surface of the first layer. A second layer of the two or more layers may include a channel-shaped-through-hole in the second layer. The first layer may be configured to receive the second layer to join the base channel and the channel-shaped-through-hole to form the channel 112 in the channel plate 110. According to various embodiments, subsequent layers of the two or more layers may include channel-shaped-through-holes in the subsequent layers. The first layer may be joined or combined with the subsequent layers to join the base channel and the channel-shaped-through-holes for form the channel 112 in the channel plate 110. The two or more layers may be joined or attached together by, but not limited to, bonding techniques such as thermal bonding, laser bonding, etc. Accordingly, in this configuration, the combination of the base channel and the channel-shaped-through-holes of the different layers may form the channel 112 with a three-dimensional channel profile in the channel plate 110 formed.

According to various embodiments, each of the base channel and the channel-shaped-through-holes of the different layers may include a channel profile different from each other. Depending on the three-dimensional channel profile required, each of the base channel and the channel-shaped-through-holes may be shaped and profiled such that when the layers are joined or attached together to form the channel plate 110, the channel 112 with the desired three-dimensional channel profile may be formed in the channel plate 110.

According to various embodiments, the channel plate 110 may include two layers. Accordingly, the groove 114 may be recessed into a surface of the second layer and configured to surround the channel-shaped-through-hole. The groove 114 may be configured to surround the channel-shaped-through-hole along a boundary of the channel-shaped-through-hole. The surface of the second layer may be an exterior surface of the formed channel plate 110. According to various embodiments, the channel plate 110 may include multiple layers. Accordingly, the groove may be recessed into a surface of the last layer and configured to surround the channel-shaped-through-hole of the last layer along a boundary of the channel-shaped-through-hole. The surface of the last layer may be an exterior surface of the formed channel plate 110.

According to various embodiments, there is provided a channel plate 110. The channel plate 110 may include a channel 112 recessed into a surface of the channel plate 110. The channel plate 110 may further include a groove 114 recessed into the surface of the channel plate 110. The groove 114 may be configured to surround the channel 112. The groove 114 may be configured to surround the channel 112 along a boundary of the channel 112. The channel plate 110 may be configured to be removably attachable to a backing plate 140 by a fastening element 150 with a substrate 130 sandwiched between the channel plate 110 and the backing plate 140 to bear a seal 120 received in the groove 114 against the channel plate 110 with the substrate 130.

According to various embodiments, there is provided a channel plate 110. The channel plate 110 may include one or more channels 112 recessed into a surface of the channel plate 110. Each of the one or more channels 112 may include a channel profile, channel features, inlets and outlets independent of other channels 112. The channel plate 110 may further include one or more grooves 114 recessed into the surface of the channel plate 110. Each of the one or more grooves 114 may be configured to surround a corresponding channel of the one or more channels 112. The groove 114 may be configured to surround the corresponding channel of the one or more channels 112 along a boundary of the corresponding channel. The channel plate 110 may be further configured to be removably attachable to a backing plate 140 by a fastening element 150 with a substrate 130 sandwiched between the channel plate 110 and the backing plate 140 to bear one or more seals received in the corresponding one or more grooves 114 against the channel plate 110 with the substrate 130.

According to various embodiments, there is provided a channel plate 110. The channel plate 110 may include two or more layers or panels joined, attached or combined together to form the channel plate 110. The channel plate 110 formed by the two or more layers or panels may include a recessed channel 112 formed into a surface of the channel plate 110. Accordingly, the channel plate 110 may include two or more layers or panels with channel features recessed into a surface of the layers or panels being combined together. The channel plate 110 may further include a groove recessed into the surface of the channel plate 110. The groove 114 may be configured to surround the channel 112. The groove 114 may be configured to surround the channel 112 along a boundary of the channel 112. The channel plate 110 may be configured to be removably attachable to a backing plate 140 by a fastening element 150 with a substrate 130 sandwiched between the channel plate 110 and the backing plate 140 to bear a seal 120 received in the groove 114 against the channel plate 110 with the substrate 130.

According to various embodiments, there is provided a flow system including the flow cell apparatus 100 as described herein, a valve connected to the channel 112 of the flow cell apparatus, and a collector connected to the valve. The valve may be a three or more way valve. The valve may be configured to direct a fluid flow through the valve into the channel 112 of the flow cell apparatus in a flow mode, and further configured to direct the fluid flow through the valve into the collector and hold the fluid in the channel 112 of the flow cell apparatus in a locked mode.

According to various embodiments, there is provided a flow system including the channel plate 110 as described herein, a valve connected to the channel 112 of the channel plate 110, and a collector connected to the valve. The valve may be a three or more way valve. The valve may be configured to direct a fluid flow through the valve into the channel 112 of the channel plate 110 in a flow mode, and further configured to direct the fluid flow through the valve into the collector and hold the fluid in the channel 112 of the channel plate 110 in a locked mode.

FIG. 1B shows a schematic diagram illustrating a method of analysing biofilm development according to various embodiments. As shown, there is provided a method 180 of analysing biofilm development. At 182, quantifying biofilm development in the flow cell apparatus as described herein or in the channel plate as described herein may be based on bio-volume. Bio-volume may be defined as the volume of the cells excluding the additional volume of the extra-cellular polymeric matrix. Accordingly, the method 180 may include quantifying biofilm development 182 in the flow cell apparatus as described herein based on bio-volume.

FIG. 1C shows a schematic diagram illustrating a method 181 of analysing biofilm development according to various embodiments. According to various embodiments, in addition to quantifying the biofilm development 182, the method 181 may further include imaging biofilm development 184 in the flow cell apparatus. Bio-volume data may be computed for each image produced from the imaging. According to various embodiments, imaging biofilm development 184 may produce three-dimensional stacked images from which bio-volume may be computed.

According to various embodiments, imaging biofilm development 184 may include imaging multiple locations along the channel of the flow cell apparatus at a specified or a predetermined time interval. For example, the specified or predetermined time interval may be 10 minutes, or may be shorter, or may be longer depending on the requirements of the experiment and the image acquisition setup. Accordingly, quantifying biofilm development 182 may include calculating bio-volume of individual biofilm clusters at each location during each imaging cycle.

According to various embodiments, the method 181 may include determining biofilm cluster distribution 186 from the quantified biofilm development. The biofilm cluster distribution may be the bio-volume of individual cluster at each location during each imaging cycle sorted in an ascending order or a descending order against the total number of clusters at that location.

According to various embodiments, the method 181 may include determining biofilm growth rate 188 from the quantified biofilm development over a period of time. The period of time may be from the start to the time at which total bio-volume reaches its maximal value. The biofilm growth rate may be calculated based on fitting the bio-volume at each location during each imaging cycle to a predetermined growth equation. An average biofilm growth rate may be calculated based on fitting the bio-volume during the period from the start to the time at which total bio-volume reaches its maximal value. According to various embodiments, the growth equation may be an exponential equation.

According to various embodiments, the method 181 may include determining biofilm removal rate 190 from the quantified biofilm development over a period of time. The period of time may be from the time right after the total bio-volume reaches its maximal value to an end time. Bio-volume at each location during each imaging cycle may be fitted into a predetermined removal equation to calculate the biofilm removal rate. An average biofilm removal rate may be calculated based on fitting the bio-volume from the time right after the total bio-volume reaches its maximal value to an end time. According to various embodiments, the removal equation may be an exponential equation.

According to various embodiments, the method 181 may further include determining the biofilm doubling time 192 from the quantified biofilm development over a period of time. The doubling time 192 may be calculated from the determined growth rate 188 based on a predetermined equation. Doubling time may be the time taken for the bio-volume to double.

Advantageously, various embodiments provided a flow cell apparatus configured to generate controllable, well-defined and reproducible environments for biofilm studies. The generation of physical and chemical environments such as shear rates and chemical gradients may be achieved by using various channel architecture, e.g. a channel having an expanded region followed by a contracted region, for a channel of the flow cell apparatus. Linear velocity gradients along the central region of the channel of the flow cell apparatus may be generated through a hyperbolic channel expansion. By infusing fluids with different chemical concentrations into the two inlets of the flow cell apparatus, well-defined chemical gradients across the width of the channel may be generated. Furthermore, decreasing chemical gradients along the central region of the channel may be generated. The flow cell apparatus may also include a removable substrate that may enable pre-treatment of the substrate surface and post-analysis of the developed biofilm on that very surface. The substrate and/or the channel may have one or more markers which may be used as spatial reference points during assembly, flow cell operation and post-analysis of biofilm developed on the substrate. The flow cell may be sealed by a gasket with a sealing seat, with an O-ring seated in an O-ring groove following the profile of the channel features. The gasket may be compressed by the channel plate containing the channel features, the removable substrate and the backing plate(s). The channel plate may be disposable and the backing plate(s) may be reusable. The channel features on the channel plate may have arbitrary two-dimensional and/or three-dimensional profile such as, but not limited to, a hyperbolic expansion. The channel plate of the flow cell apparatus may contain one or more channels. Each of the channels may have their independent channel features. Furthermore, each of the channels may have their independent seals using a gasket with a sealing seat, with O-rings seated in O-ring grooves being particularly preferred. In addition, the inflows and outflows of each channel may be independent. The channel plate may include two or more layers or panels with channel features. The layers may be attached together by, but not limited to, bonding techniques such as thermal bonding, laser bonding, etc. Sealing and flatness of the observed surface in the flow cell may be enhanced by the use of the backing plate(s). The rim of the observation window on the primary backing plate may be tapered to improve the accessibility of observation/measurement tools (such as microscope objective lens) to regions of interest on the substrate. A winged backing plate (a backing plate having one or more wings added to its sides, with two wings generally being preferred) may be used to ensure firm clamping of the device during measurement and analysis, e.g. fixing on microscope sample holder. The compression force may be supplied by fasteners such as, but not limited to, bolts and nuts, snapfit fasteners (such as snap-lock pins) together with compression springs, or clamps.

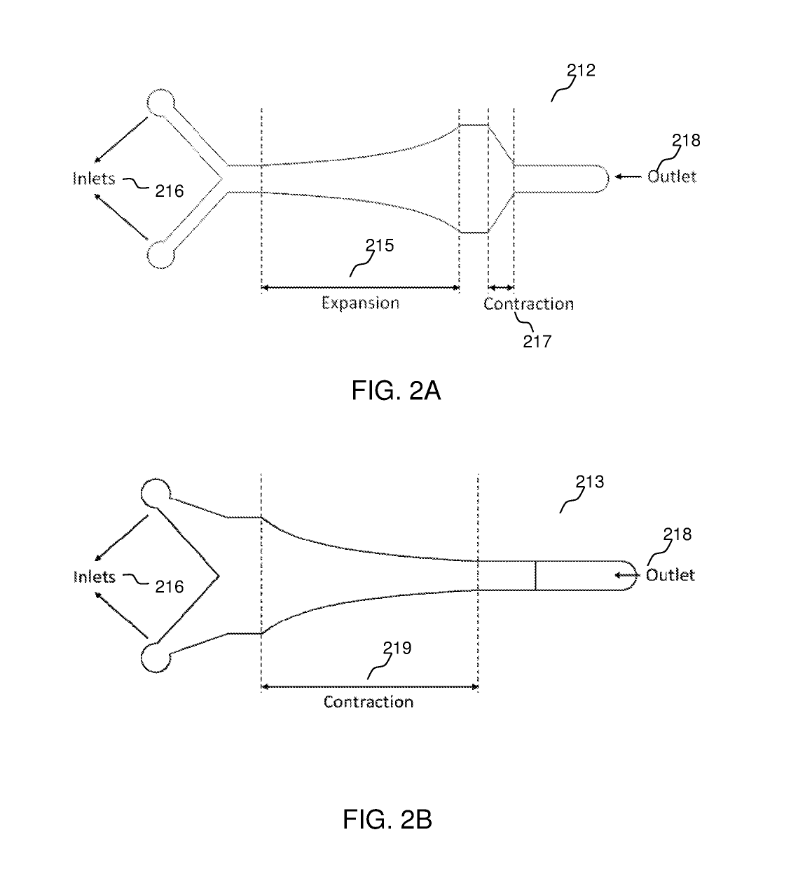

FIGS. 2A and 2B show channels 212, 213 which may generate a controllable, well-defined and reproducible environment for biofilm studies. FIG. 2A shows the channel 212 with a hyperbolic expansion. FIG. 2B shows the channel 213 with a hyperbolic contraction. As shown, the channels 212, 213 may include at least one inlet 216, and generally preferred to have at least two inlets 216, feeding into the respective channels which lead into one or more outlets 218, but generally preferred to have one outlet. In FIG. 2A, the channel 212 is shown to have an expanded region 215 which may generate a decreasing shear rate gradient, followed by a contracted region 217. In FIG. 2B, the channel 213 is shown to have a contracted channel 219 for the generation of increasing shear rate gradient. The number of inlets 216 may not be limited to two, as illustrated in FIGS. 2A and 2B, but may be extended to any desired number. The infusion of fluids into the inlets 216 may be independently controlled by different pumps. By having one outlet 218, all the fluids infused into the flow cell may converge into the outlet 218. Therefore, the flow field in the flow cell may be independent of the peripheral connections such as tubing and other devices. Although more than one outlet 218 may be possible, it may not be desirable as special arrangement may be necessary for precise control of flow field in the flow cell. With a characteristic dimension in the order of 100 μm to millimeters, the flow cell may operate in laminar flow regime such that fluidic conditions in the channel may be controllable, well-defined and reproducible. The platform may also enable the micro-scaled control of the environment to generate physical and chemical micro-environments.

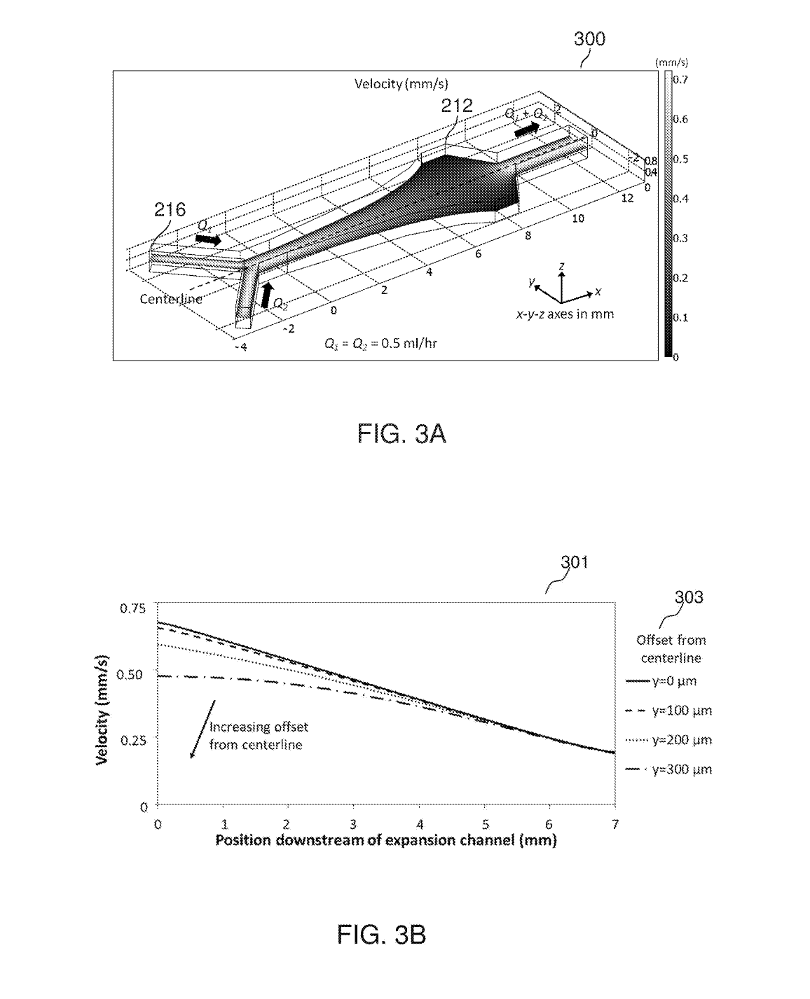

FIGS. 3A and 3B show representations 300, 301 of the controllable velocity field in the channel 212 of FIG. 2A in which a hyperbolic expansion is used to generate a linear shear rate gradient in the center of the channel 212 according to various embodiments. FIG. 3A shows a schematic illustration 300 of the xy-midplane velocity along the channel 212. FIG. 3B shows a line graph 301 illustrating lines representing velocity along various paths 303 offset from the centreline in the channel 212. As shown in FIGS. 3A and 3B, by using hyperbolic expansion as the expanded region, a linear shear rate gradient in the vicinity of the expanded region's centerline of the channel 212 may be generated. As shown in FIG. 3B, by infusing fluids into the two inlets 216 with the same rate, velocity over ±200 μm wide zone from the centerline of channel 212 may be linearly decreasing.

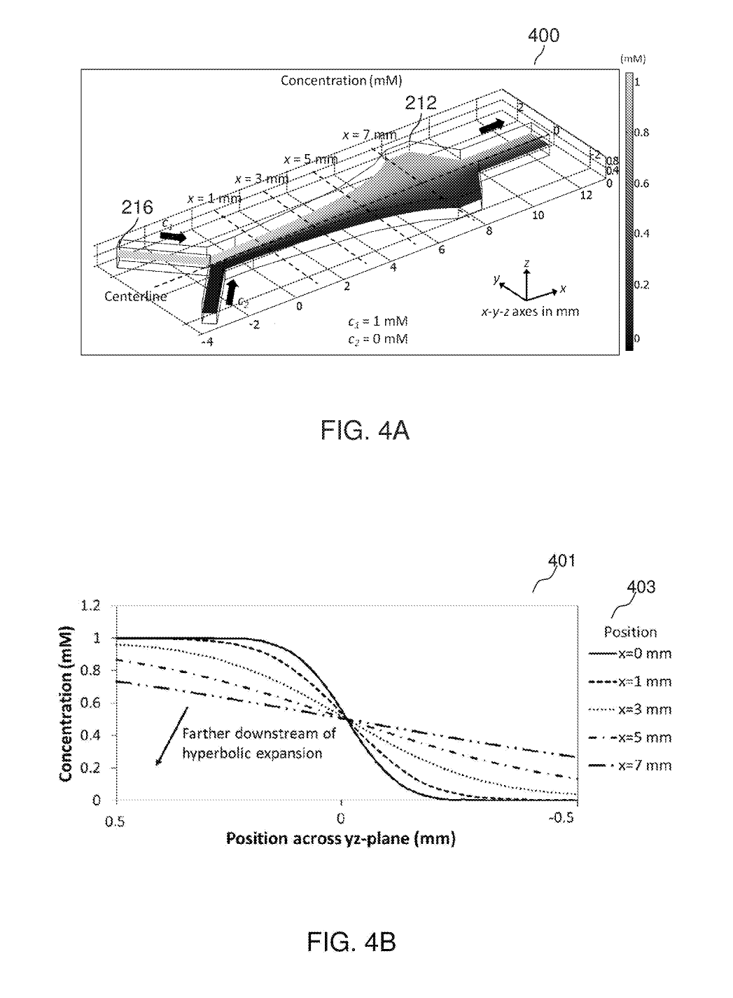

FIGS. 4A and 4B show representations 400, 401 of the controllable concentration in the channel 212 in which a hyperbolic expansion may be used according to various embodiments. FIG. 4A shows a schematic illustration 400 of the xy-midplane concentration along the channel 212. FIG. 4B shows a line graph 401 illustrating lines representing concentration across the hyperbolic expansion at various positions 403 along the channel 212. As shown in FIGS. 4A and 4B, by infusing fluids of different chemical concentrations into the two inlets 216 respectively, a well-defined chemical gradient across the width of the channel 212 may also be generated. As shown in FIG. 4B, the chemical gradient at the centerline of the expanded region decreases along the channel 212 may give rise to the second order derivative of chemical concentration.

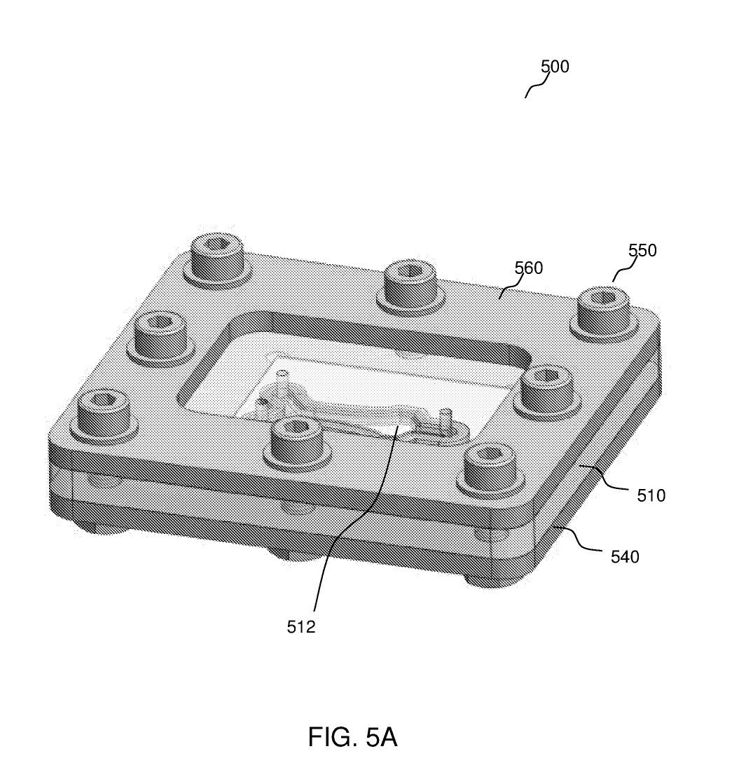

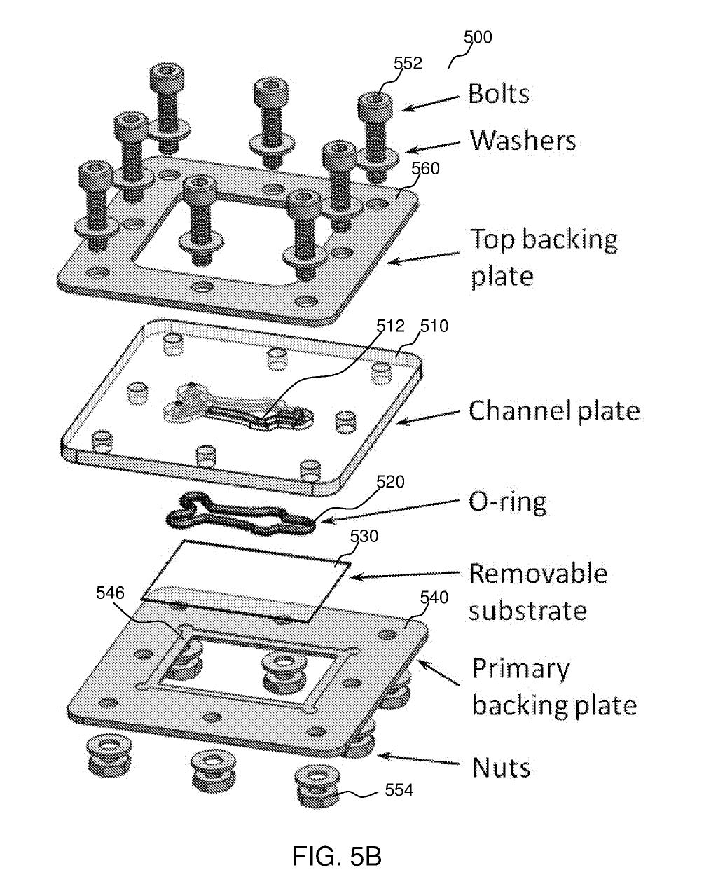

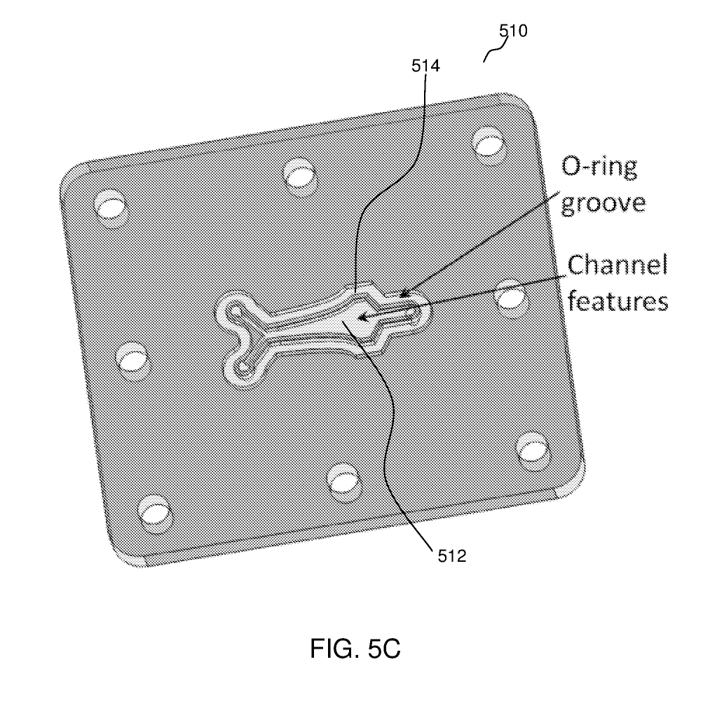

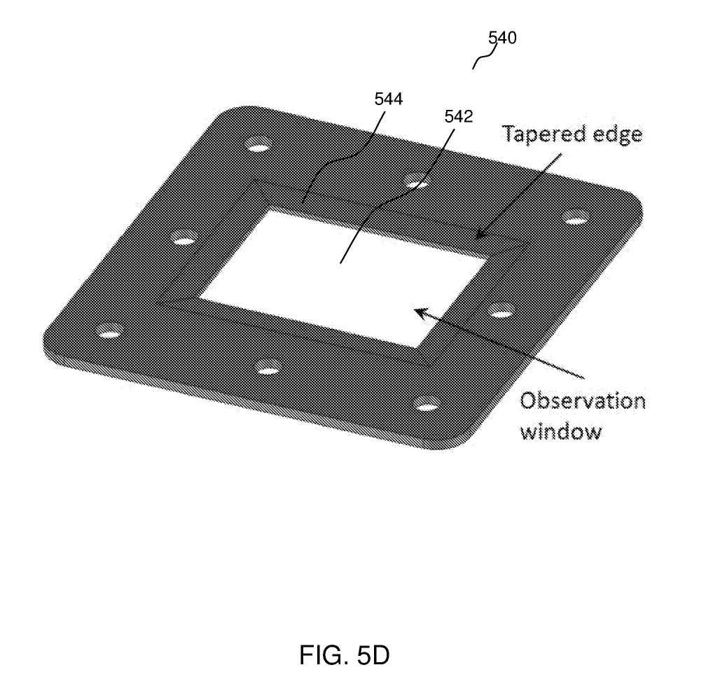

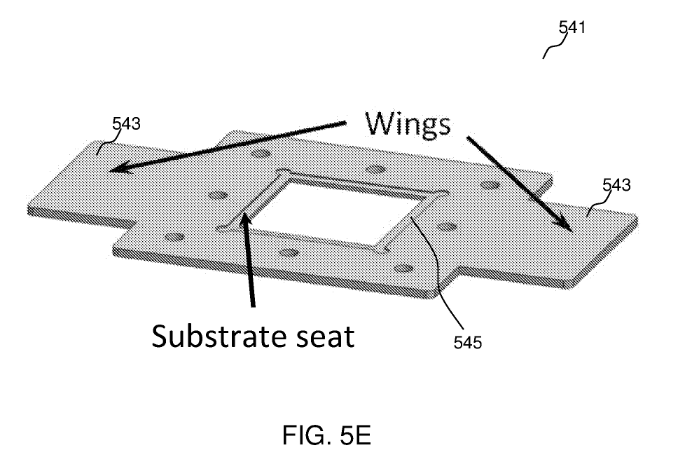

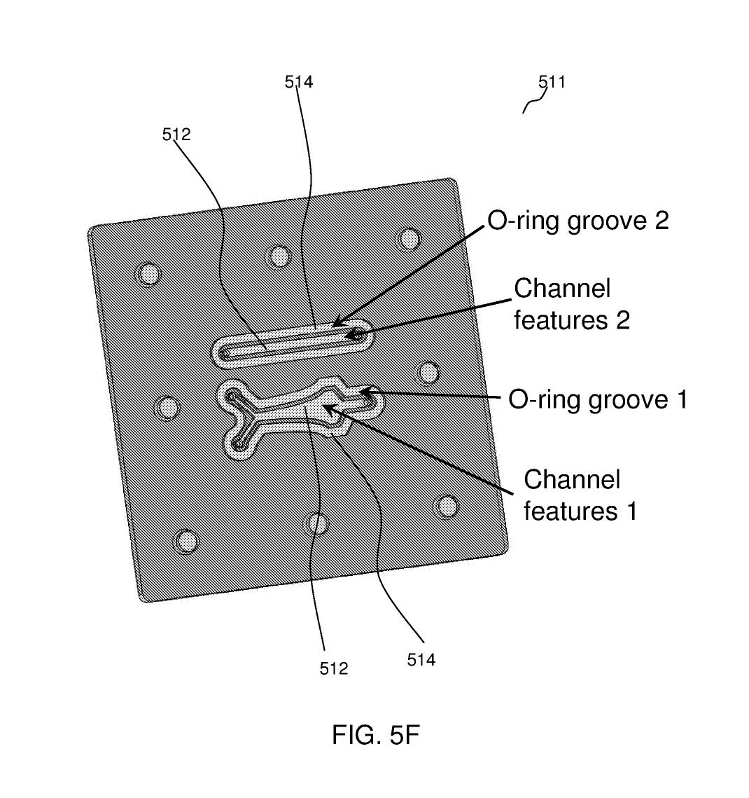

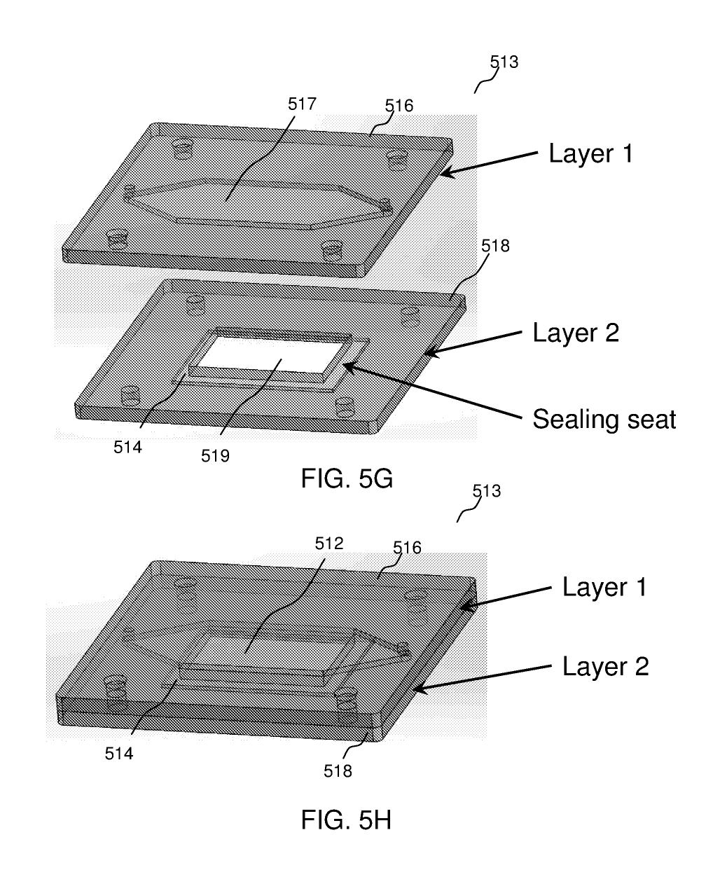

FIG. 5A shows an assembled flow cell apparatus 500 according to various embodiments. FIG. 5B shows an exploded view of the flow cell apparatus 500 according to various embodiments. FIG. 5C shows a channel plate 510 of the flow cell apparatus 500 of FIG. 5B according to various embodiments. FIG. 5D shows a backing plate 540 of the flow cell apparatus 500 of FIG. 5B according to various embodiments. FIG. 5E shows a backing plate 541 according to various embodiments. FIG. 5F shows a channel plate 511 according to various embodiments. FIG. 5G shows an exploded view of a channel plate 513 according to various embodiments. FIG. 5H shows an assembled view of the channel plate 513 according to various embodiments.

FIGS. 5A and 5B illustrate the flow cell apparatus 500 which may generate controllable, well-defined and reproducible environments for biofilm research. The flow cell apparatus 500 may include a channel plate 510, a seal 520 (e.g. a gasket or an O-ring), a removable substrate 530, a primary backing plate 540, with or without at least one other backing plate (e.g. top backing plate 560), and fasteners 550. As shown in FIGS. 5A and 5B, two backing plates 540, 560 together with bolts 552 and nuts 554 as fasteners 550 may be used. The removable substrate 530 may be a microscope coverslip, a polymer sheet/film, etc. The solid surface of the removable substrate 530, on which the biofilm grows, may be subjected to pre-treatment before being assembled into the flow cell apparatus 500. The removable substrate 530 may also be removed at any point of the experiment, with the biofilm intact, for post-analysis. The substrate 530 and/or the channel 512 may include at least one marker (not shown) on the surfaces, with minimum three markers being generally preferred, for using as spatial reference points during assembly, flow cell operation and post-analysis of biofilm on the substrate 530. The channel plate 510 shown in FIG. 5C with a hyperbolic expansion used as the channel profile as an example, which may be disposable, may contain the channel features 512 and the sealing seat 514 (e.g. a groove or an O-ring groove). The channel features 512 may have arbitrary two-dimensional and/or three-dimensional profile. The gasket 520 seated in the sealing seat 514, particularly an O-ring seated in an O-ring groove following the profile of the channel features 512 as shown in FIGS. 5B and 5C, may be used to ensure that the flow cell apparatus 500 may be leak-proof and may have good dimensional tolerance. Sealing may be achieved by compressing the gasket 520 and the removable substrate 530 when the substrate 530 is sandwiched between the channel plate 510 and the primary backing plate 540. The primary backing plate 540 may include a recessed portion 546 to receive the removable substrate 530. The backing plate 540 may ensure the flatness and may provide rigidity to the assembled flow cell apparatus 500. One or more additional backing plates 560 may be added to provide additional support. The backing plate(s) 540, 560, which may be reusable, may be a flat plate made of materials such as stainless steel or polymer. The primary backing plate 540 may contain an observation window 542, with edges 544 that may be tapered as shown in FIG. 5D to improve the accessibility of the observation and/or measurement tools such as, but not limited to, the lens of a microscope, to regions of interest on the substrate 530.

According to various embodiments, as shown in FIG. 5E, one or more wings 543, with two wings being preferred, may be added to the sides of the backing plate to form a winged backing plate 541 to enhance firm clamping of the flow cell apparatus 500 during operation, such as measurement and analysis. For example, the winged backing plate may enhance fixing of the flow cell apparatus 500 on microscope sample holder. The backing plate 541 may include a recessed portion 545 to receive the removable substrate 530.

According to various embodiments, fasteners 550, such as bolts 552 and nuts 554, snapfit fasteners with compression springs, may be used to provide the compression force. The exact number of the fasteners 550 may be varied, with four fasteners being generally preferred. Using bolts 552 and nuts 554 as the fasteners 550, as illustrated in FIG. 5B, would advantageously require, although not absolutely necessary, the use of torque wrench and washers during assembly to ensure accurate and even stress distribution over the flow cell apparatus 500 and thus the sealing.

FIG. 5F shows a channel plate 511 that may include one or more channels 512 according to various embodiments. The channel plate in FIG. 5F may include two channels 512, for example Channel Features 1 and Channel Features 2. The two channels may be recessed into the surface of the channel plate 511. The two channels 512 may have independent channel features (Channel Features 1 and Channel Features 2), inflows and outflows, sealing seats 514 (O-ring Groove 1 and O-ring Groove 2) and seals. Accordingly, each of the two channels 512 may include a channel profile different from each other. Each of the sealing seats 514 (or grooves) may be configured to surround a corresponding channel of the two channels 512. Each of the sealing seats 514 may be configured to surround the corresponding channel of the two channels 512 along the boundary of the corresponding channel. Thus, the each channel of the two channels 512 may have a sealing seat 514 following the profile of the channel. As shown in FIG. 5F, the channels on a channel plate may be, but not limited to, a straight channel and a hyperbolic expansion channel. By having two or more channels enable multiplexed experiments to be conducted simultaneously on a single flow cell apparatus. According to various embodiments, the channel plate 511 may include two or more channels 512.

FIGS. 5G and 5H show a channel plate 513 that may include two or more layers or panels. FIG. 5G shows an exploded view of the channel plate 513 and FIG. 5H shows an assembled view of the channel plate 513. FIGS. 5G and 5H show the formation of three-dimensional channel 512 features by combining two layers 516, 518 that have channel features 517, 519. The two layers 516, 518 (e.g. Layer 1 and Layer 2) may be combined by, but not limited to, bonding techniques. Each of the two layers 516, 518 may have different channel features 517, 519, and their combination may give three-dimensional features to the channel 512. For example, the channel features 517 in the first layer 516 (Layer 1) may include a base channel with a first profile, and the channel features 519 in the second layer 518 (Layer 2) may include a channel-shaped-through-hole with a second profile that is different from the first profile. In addition, layer 518 (Layer 2) may include a sealing seat 514 (or groove) for the seal. The sealing seat 514 may be recessed into a surface of the layer 518 (Layer 2) and be configured to surround the channel features 519. The sealing seat 514 may be configured to surround the channel features 519 along a boundary of the channel features 519. According to various embodiments, the channel plate 513 may include two or more layers.

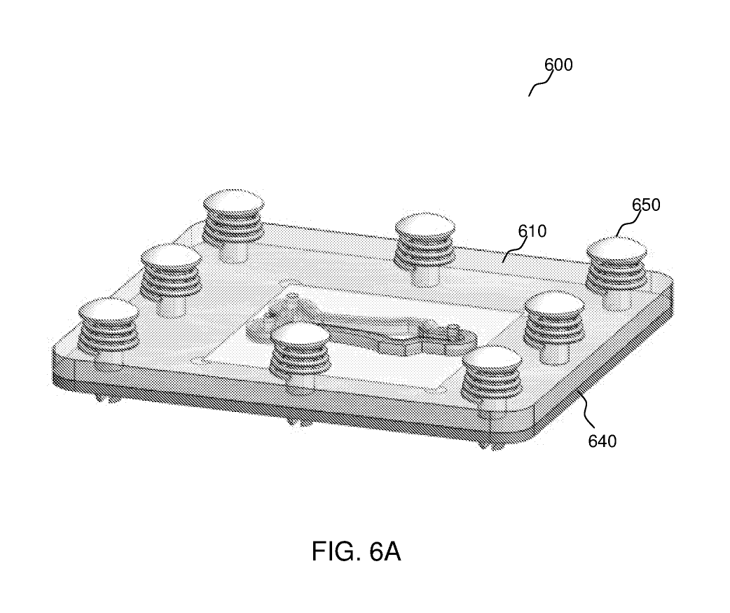

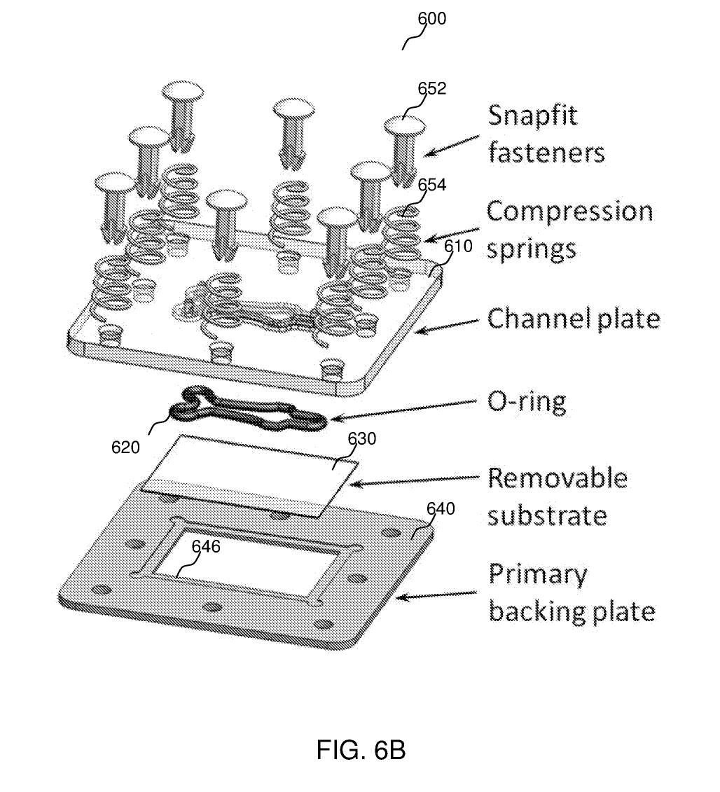

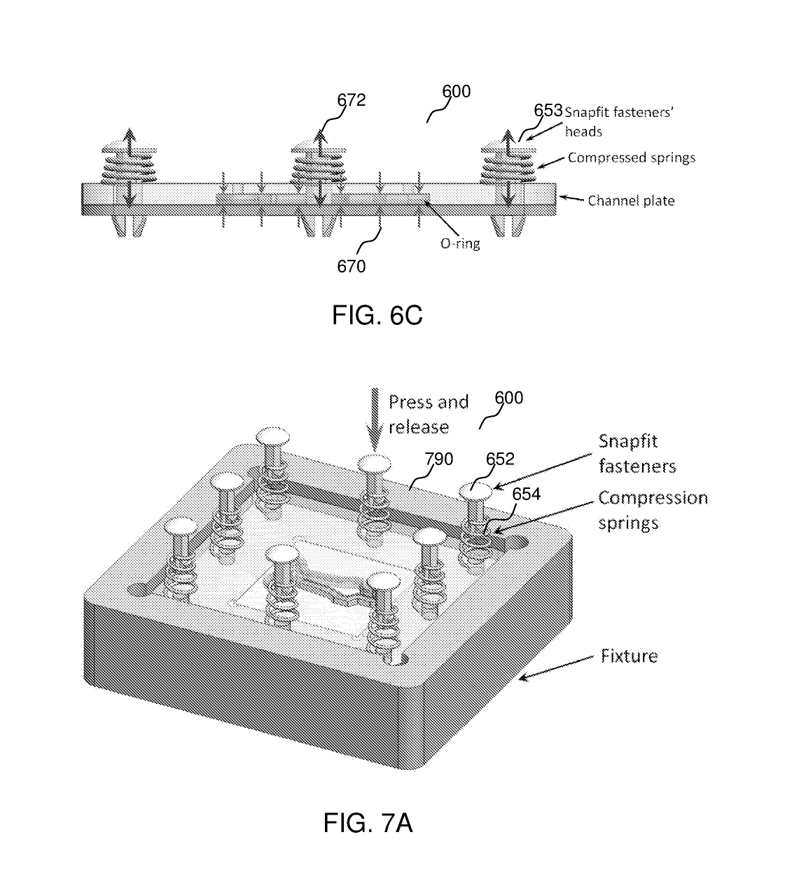

FIG. 6A shows an assembled flow cell apparatus 600 according to various embodiments. FIG. 6B shows an exploded view of the flow cell apparatus 600 according to various embodiments. FIG. 6C shows a side view of the assembled flow cell apparatus 600 according to various embodiments.

As shown in FIGS. 6A, to 6C, the flow cell apparatus 600 may include only one backing plate 640 (i.e. primary backing plate), and with snapfit fasteners 652 and compression springs 654 to replace the bolts 552 and nuts 554 in the flow cell apparatus 500 as shown in FIGS. 5A and 5B. The flow cell apparatus 600 may include a removable substrate 630, a channel plate 610, a seal 620 (e.g. a gasket or an O-ring), the primary backing plate 640, the snapfit fasteners 652 and the compression springs 654. The removable substrate 630, the channel plate 610 and the backing plate 640 may be similar to the removable substrate 530, the channel plate 510 and the backing plate 540 in FIGS. 5A and 5B. The backing plate 640 may include a recessed portion 646 to receive the removable substrate 630. The snapfit fasteners 652 (such as snap lock pins) together with the compression springs 654 may be used to provide the necessary sealing force and function as a set of fastener 650. The exact number of the fasteners 650 may be varied, with four fasteners 650 being generally preferred. As shown in FIG. 6C, the compressive stress 670 applied over the O-ring 620 may be self-balanced by the resistance 672 of the compression springs 654 as the compression springs 654 are compressed and constrained by the snapfit fasteners' heads 653 and the channel plate 610. In addition, any variation in the plates' thicknesses due to fabrication process may be compensated and thus may have no effects on the sealing quality as the sealing force required is self-adjusted by the compression springs 654.

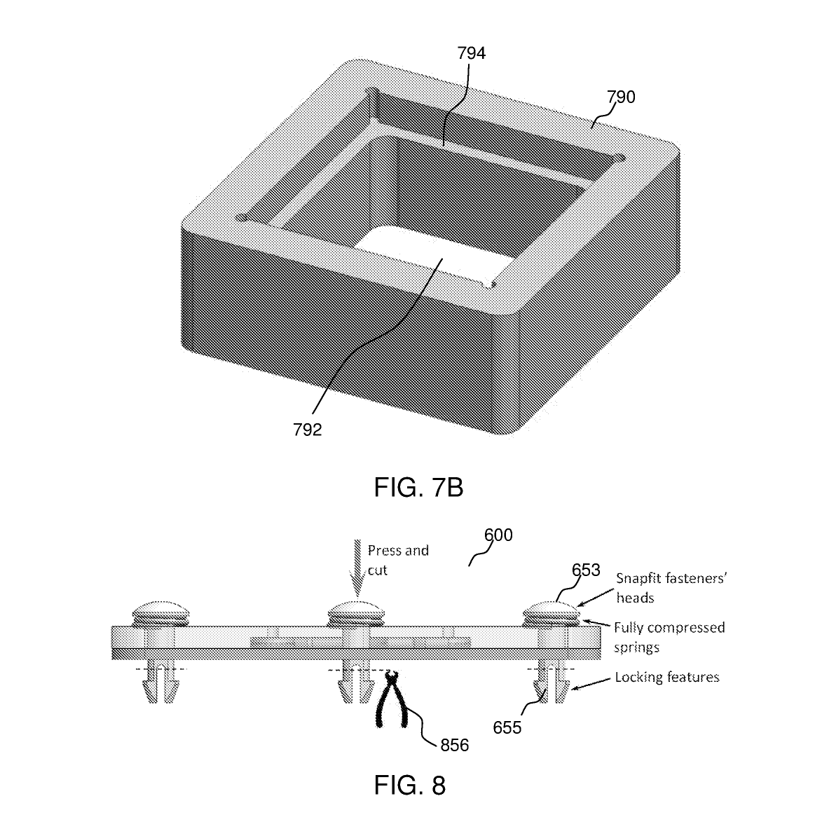

FIG. 7A shows a schematic diagram of assembling the flow cell apparatus 600 with the aid of a fixture 790 according to various embodiments. FIG. 7B shows the fixture 790 of FIG. 7A according to various embodiments. FIG. 7A illustrates the use of snapfit fasteners 652 and compression springs 654 to simplify the assembly of the flow cell apparatus 600. The combination of snapfit fasteners 652 and compression springs 654 may enable the self-locked part-to-part attachment of the assembled flow cell apparatus 600, which may allow fast assembly via “press-and-release”. As shown in FIG. 7A, with the aid of the fixture 790, quick assembly may be enabled by placing the flow cell's components inside the fixture 790, followed by pressing-and-releasing the heads 653 of snapfit fasteners 652. Accordingly, FIG. 7A demonstrated the “press-and-release” assembly method by using the snapfit fasteners 652 and the compression spring 654 with the aid of the fixture 790. FIG. 7B is an illustration of the fixture 790 which may provide rigid support to facilitate quick assembly. As shown, the fixture 790 may include a through hole 792 and a ledge 794 in the through hole 792. The flow cell's components, such as the backing plate 640 and the channel plate 610, with the substrate 630 sandwiched between, may be placed in the through hole 792 such that the backing plate 640 may rest on the ledge 794. The snapfit fasteners 652 and the compression spring 654 may then be “press-and-release” into the flow cell's components to assemble the flow cell apparatus 600.

FIG. 8 shows a schematic diagram of dismantling the flow cell apparatus 600 according to various embodiments. FIG. 8 illustrates the use of snapfit fasteners 652 and compression springs 654 to simplify the dismantling of the flow cell apparatus 600. As shown, the combination of snapfit fasteners 652 and compression springs 654 may allow quick “press-and-cut” disassembly of the flow cell apparatus 600 by pressing the heads 653 of the snapfit fasteners 652 to fully compress the springs 654, followed by cutting the locking features 655 with a cutter 856 such as a side cutting pliers. Accordingly, FIG. 8 demonstrated the “press-and-cut” disassembly method by pressing the snapfit fasteners' heads 653 and cutting the locking features 655 of the snapfit fasteners 652 with the cutting pliers 856.

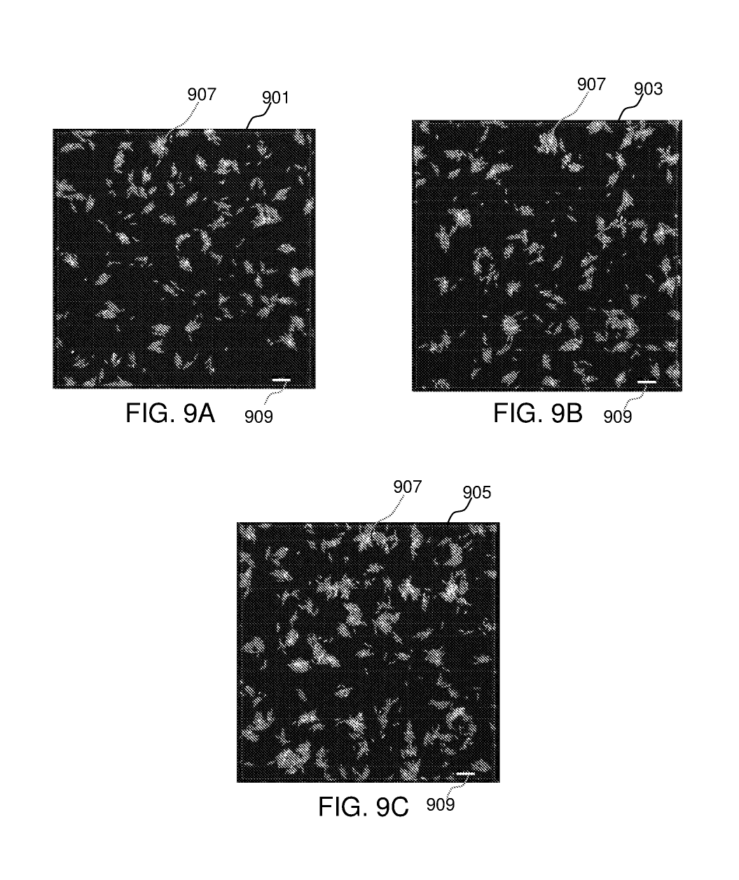

FIGS. 9A to 9C show confocal microscopy images, 901, 903, 905 of monospecies biofilm formed after 3.5 hours growth at room temperature (25° C.) at various locations along the hyperbolic expansion of the channel 212 as shown in FIG. 3A according to various embodiments. The bacteria strain was Pseudomonas putida OUS82::YFP (yellow 907). The images were obtained by using confocal laser scanning microscopy with 40× magnification. The bacteria strain used was fluorescently tagged with yellow fluorescent protein 907. FIG. 9A shows the confocal microscopy image 901 at the start of the hyperbolic expansion. FIG. 9B shows the confocal microscopy image 903 at the central of the hyperbolic expansion. FIG. 9C shows the confocal microscopy image 905 at the end of the hyperbolic expansion. In

FIGS. 9A, to 9C, all the scale bars 909 are 15 μm.

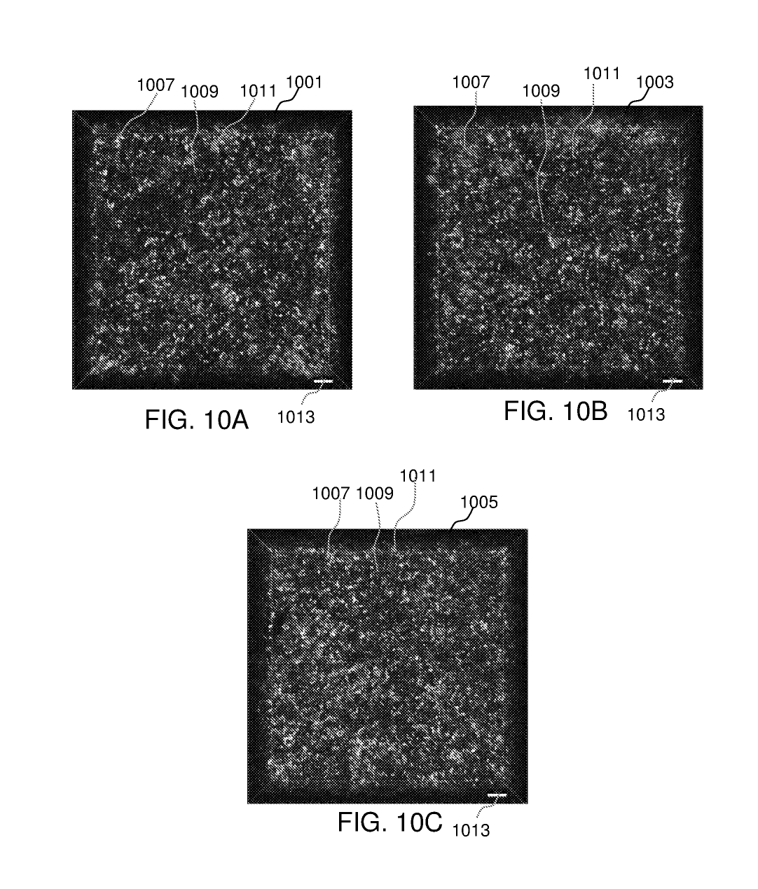

FIGS. 10A, 10B and 10C show confocal microscopy images 1001, 1003, 1005 of a 3-day-old multispecies biofilm cultured at room temperature (25° C.) at different positions along the hyperbolic expansion of the channel 212 as shown in FIG. 3A according to various embodiments. The mixed-species biofilms include three species, particularly Pseudomonas aeruginosa (PAO1, yellow 1007), Pseudomonas protegens (Pf-5, cyan 1009) and Klebsiella pneumonia (KP-1, red 1011). Each of them was fluorescently tagged with different colors 1007, 1009, 1011 to facilitate colocalization. The confocal microscopy images 1001, 1003, 1005 were captured by using confocal laser scanning microscopy with a magnification of 63×. FIG. 10A shows the confocal microscopy image 1001 at the start of the hyperbolic expansion. FIG. 10B shows the confocal microscopy image 1003 at the central of the hyperbolic expansion. FIG. 10C shows the confocal microscopy image 1005 at the end of the hyperbolic expansion. The scale bar 1013 in each image 1001, 1003, 1005 is 10 μm.

Various embodiments provided a flow cell that may generate controllable, well-defined and reproducible environments for biofilm studies. The environments may include, but not restricted to, physical and chemical factors. Physical parameters may include, but not exclusive to, hydrodynamic stresses and temperature. Chemical factors may include, but not limited to, chemical concentration, gas concentration, surface energy and wettability of the substrate and the channel walls. The flow cell may have at least one inlet, and generally may be preferred to have two or more inlets for feeding into a channel that leads into one outlet or more. It may be preferred, although not limited, to have one outlet. The channel profile may be changeable for the generation of physical and chemical environments, such as but not exclusively confined to, shear rate and chemical gradients by employing an expanded region followed by a contracted region. A hyperbolic expansion, for example, may be used to generate linear shear rate gradients in the vicinity of the expanded channel's centerline. By infusing fluids and/or gas with different chemical concentrations into the two inlets respectively, well-defined chemical gradients across the width of the channel may also be generated. Furthermore, the second order derivative of the chemical concentration, in the form of decreasing chemical gradients, may also arise in the central region along the channel.

The infusion of fluids into the inlets of the flow cell may be independently controlled by different pumps. Thus, a combination of different fluids and flow rates may be infused into the respective inlets to enable the control over the environment in the flow cell. In addition, by having one outlet, all the fluids infused into the flow cell may converge into the outlet. Therefore, the flow field in the flow cell may be independent of the peripheral connections such as tubing and other devices. Although more than one outlet may be possible, it may not be desirable as special arrangement may be necessary for precise control of flow field in the flow cell. With a characteristic dimension in the order of 100 μm to millimeters, the flow cell may operate in laminar flow regime such that fluidic conditions in the channel may be controllable, well-defined and reproducible. The platform may also have micrometer resolution in the control of the environment to generate physical and chemical micro-environments. The well-defined environments in the flow cell may be predicted by fluid dynamics simulation and may be verified experimentally.

In addition to the fluidic environments, the substrate, which may be the solid surface/interface on which the biofilm grows, may also be controlled in this flow cell. The substrate in the flow cell may be removable. The removable substrate may lend itself to pre-treatment of its surface and post-analysis of the developed biofilm. The substrate may be, but not exclusive to, a microscope coverslip or polymer sheet/film. The substrate may be subjected to pre-treatment before being assembled into the flow cell such as, but not limited to, surface modification, surface patterning and texturing, coating, etc. Furthermore, the substrate may be removed at any point of the experiment, with the biofilm intact, for downstream analysis such as, but not limited to, meta-omics analyses, atomic force microscopy, scanning electron microscopy, etc. The substrate and/or the channel plate may have one or more markers on the surfaces to be used as spatial reference locations such as, but not exclusive to, during flow cell assembly and operation, and post-analysis of biofilm on the substrate. It may be generally preferred to have at least three markers. The markers may be made by, but not exclusive to, laser marking, etching or machining.

As the substrate may not be permanently bonded onto the flow cell, precision sealing may be important to ensure that the flow cell may be leak-proof and may have good dimensional tolerance. The flow cell may be sealed using a gasket seated in a sealing seat, with an O-ring seated in an O-ring groove following the profile of the channel features being particularly preferred. The flow cell may include a channel plate containing the channel features and the sealing seat (e.g. an O-ring groove following the profile of the channel features), a gasket (e.g. an O-ring), a removable substrate, a primary backing plate, with or without at least one additional backing plate(s), and fasteners. The substrate may be placed in the seat on the primary backing plate for alignment of the substrate and fast assembly of the flow cell. The primary backing plate may also contain a window for observations, the edges of which may be tapered to enhance the accessibility of the observation or measurement tools. The channel plate may be disposable and the backing plate(s) may be reusable. The channel features on the channel plate may have arbitrary two-dimensional and/or three-dimensional profile such as, but not limited to, a hyperbolic expansion. Sealing may be achieved by compressing the gasket and the substrate when the substrate is sandwiched between the channel plate and the primary backing plate. The backing plate may ensure flatness and may provide rigidity to the assembled flow cell. Additional backing plate(s) may be added to provide additional support.

According to various embodiments, the channel plate of the flow cell apparatus may contain one or more channels. Each of the channels may have their independent channel features. Furthermore, each of the channels may have their independent seals using a gasket with a sealing seat, with O-rings seated in O-ring grooves being particularly preferred. In addition, the inflows and outflows of each channel may be independent. The environments in the channels, which may include, but not limited to, flow fields and chemical conditions, may be independently controlled. Therefore, multiplexed experiments may be conducted simultaneously on a single flow cell apparatus. According to various embodiments, the channel plate may consist of two or more layers or panels with channel features. The layers may be attached together by, but not limited to, bonding techniques such as thermal bonding, laser bonding, etc. The combination of features on different layers may enable the formation of three-dimensional features in the channel.

According to various embodiments, the backing plate in the form of a winged backing plate may be used to facilitate firm clamping of the device during operation, e.g. placing on the stage holder of microscope. The winged backing plate may have one or more wings added to its sides, with two wings generally being preferred. The backing plate(s) may be a flat plate made of materials such as, but not limited to, stainless steel or polymer. The compression force may be provided by fasteners such as, but not limited to, bolts and nuts, snapfit fasteners and compression springs.

Using bolts and nuts as the fasteners may require the use of torque wrench and washers during assembly to ensure accurate and even stress distribution over the flow cell and thus the sealing. In order to simplify the assembly and dismantling of the disclosed platform without compromising proper sealing, snapfit fasteners (such as snap-lock pins) together with compression springs may be used to provide the necessary sealing force. The combination of snapfit fasteners and compression springs may enable the self-locked part-to-part engagement of the assembled flow cell, which may allow fast “press-and-release” assembly. The compressive stress required to be applied over the O-ring for proper sealing may be self-balanced by the resistance of the compression springs as the springs are compressed and constrained by the snapfit fasteners' heads and the plate. In addition, any variation in the plates' thicknesses due to fabrication process may be compensated and thus may have no effects on the sealing quality as the sealing force required is self-adjusted by the compression springs. The combination of snapfit fasteners and compression springs may also contribute to quick “press-and-cut” disassembly of the flow cell by pressing the heads of the snapfit fasteners to fully compress the springs, followed by cutting the locking features with a cutter such as a side cutting pliers.

The assembly and dismantling of the flow cell may be operated manually or with the aid of a fixture. The disclosed fixture which provides a rigid support may be configured to enhance further the quick assembly or disassembly of the flow cell.

The flow cell may be configured to generate controllable well-defined and reproducible environments for biofilm research. In addition to the functional aspect, the operation of the flow cell may also be robust and simple to execute.

Various embodiments may be defined by the following numbered clauses.

Example 1 is a flow cell that may function as a platform for biofilm studies for both monospecies and multispecies that includes biofilm culture and experiment. The flow cell may generate controllable, well-defined and reproducible environments. Controllable environments include physical and chemical factors. For example, (i) the generation of shear rate gradients. Decreasing shear rates by an expanded region followed by a contracted region. Increasing shear rate gradients by a contracted channel. (ii) The generation of thermal gradients. (iii) The generation of chemical gradients. (iv) The generation of second order derivative of chemical concentration. (v) The ability to modify the surface properties of channels' walls.

In Example 2, the subject matter of Example 1 may further include the generation of linearly decreasing shear rate gradients by a hyperbolic expanded region followed by contracted region, and the generation of linearly increasing shear rate gradients by a hyperbolic contracted channel.

In Example 3, the subject matter of Example 1 or 2 may further include the generation of chemical gradients through infusion of liquids of different chemical concentrations and flow rates into the two or more inlets of the flow cell.

In Example 4, the subject matter of any one of Examples 1 to 3 may further include the control of the environment with micrometer resolution to generate physical and chemical micro-environments.

In Example 5, the subject matter of any one of Examples 1 to 4 may further include the control of the change of the environment with a controlled change in the flow rate and/or the inlet fluid conditions such as temperature and/or chemical concentration.

Example 6 is a flow cell that includes a channel plate containing channel features and a sealing seat, a gasket, a removable substrate, a primary backing plate that contains the seat for the removable substrate and a window for observations, with or without at least one additional backing plate, and fasteners. The edges of the observation window may be tapered to enhance the accessibility of the observation tools.

In Example 7, the subject matter of Example 6 may include that the channel plate may have one or more channels on the same channel plate. Each of the channels may have independent channel features, seals, inflows and outflows.

In Example 8, the subject matter of Example 6 or 7 may include that the channel plate may be formed by combining two or more layers or panels that contain channel features.

In Example 9, the subject matter of any one of Examples 6 to 8 may include that the channel plate may be disposable. The channel features on the channel plate may have arbitrary two-dimensional and/or three-dimensional profile such as, but not limited to, a hyperbolic expansion. The sealing seat on the channel plate may be generally preferred, but not limited to, an O-ring groove preferably following the profile of the channel features.

In Example 10, the subject matter of any one of Examples 6 to 9 may further include that the removable substrate may be configured to enable pre-treatment of the substrate and post-analysis of the developed biofilm on the substrate. This removable substrate and/or the channel may further have at least one marker, with minimum three markers being generally preferred, for using as spatial reference locations such as, but not limited to, during flow cell assembly and operation, and post-analysis of the biofilm developed on the substrate.

In Example 11, the subject matter of any one of Examples 6 to 10 may further include a sealing method by a gasket seated in a sealing seat, with an O-ring seated in an O-ring groove preferably following the profile of the channel features being particularly preferred, in which sealing may be achieved by compressing the gasket (such as the O-ring) with the substrate, the channel plate and the backing plate(s).

In Example 12, the subject matter of Example 11 may further include the use of backing plate(s), i.e. one primary backing plate with/without at least one additional backing plate, to ensure flatness and provide rigidity to the assembly of the flow cell in Example 6. The backing plate(s) may be reusable. A winged backing plate, i.e. a backing plate with one or more wing(s) may be added to its sides with two wings generally preferred, may be used as an another configuration to enhance firm clamping of the device during its operation such as clamping on microscope sample holder for imaging.

In Example 13, the subject matter of Example 11 or 12 may further include using fasteners, such as bolts and nuts, tightened by a torque wrench (advantageously required, although not absolutely necessary) to ensure repeatable and uniform stress distribution over the sealing area.

In Example 14, the subject matter of Example 11 or 12 may further include using snapfit fasteners such as snap-lock pins together with compression springs, as another configuration to the fasteners in Example 11.

In Example 15, the subject matter of Example 14 may further include the ability to achieve self-locked part-to-part engagement of the assembled flow cell, which may allow fast “press-and-release” assembly.

In Example 16, the subject matter of Example 14 or 15 may further include the ability to achieve self-balancing of compressive stress applied over the sealing area such that the stress distribution is repeatable and even.

In Example 17, the subject matter of any one of Examples 14 to 16 may further include the ability to compensate for any variation in the plates' thicknesses due to fabrication process to ensure sealing quality.

In Example 18, the subject matter of any one of Examples 14 to 17 may further include the ability of quick “press-and-cut” disassembly of the flow cell by pressing the heads of the snapfit fasteners to fully compress the springs, followed by cutting the locking features with a cutter such as a side cutting pliers.

In Example 19, the subject matter of any one of Examples 1 to 18 may include further enhancing the ability of quick assembly and disassembly of the flow cell with the aid of a fixture. The fixture may contain a seat for the flow cell's parts, particularly the backing plate and the channel plate, for fast alignment of these parts during assembly of the flow cell. The fixture may also provide rigid support during the “press-and-release” assembly.

A case study of observation of biofilm development conducted under well-defined environments will be described in the following.

In this case study, a high spatio-temporal resolution approach for the real-time study of biofilm behaviour under well-controlled environmental conditions will be described. According to various embodiments, a flow cell was designed and fabricated to create well-defined and reproducible environments. The flow cell was fabricated by micro-machining processes that were optimized for precision and reproducibility. The chamber has a removable substrate (microscopy glass coverslip) that allows for pre-treatments of the surface by various surface modifications and for downstream analyses of the intact biofilm; these features are not supported in any other presently available flow cell.

By employing confocal laser scanning microscopy, long-term, high content live imaging at multiple locations in the flow cell is demonstrated. Any specific position of interest inside the chamber can be revisited at any time of the experiments with an accuracy of ±2 μm. The accuracy of the positioning is only limited by the accuracy of the motorized stage of the microscope. A protocol to operate the flow cell including simulation and validation of flow field, biofilm experiments and quantitative biofilm growth analysis is presented. It is shown and quantified for the first time the unpredicted dynamic formation and dispersal of a model biofilm using a well-documented strain of Pseudomonas putida under controlled shear condition at multiple positions in the chamber with a 10-minute interval over 8 h 20 min. Only when applying high spatial and temporal resolutions, cycles of growth and dispersal behaviours can be observed during initial biofilm development.

The set-up of flow cell system used will be described in the following.

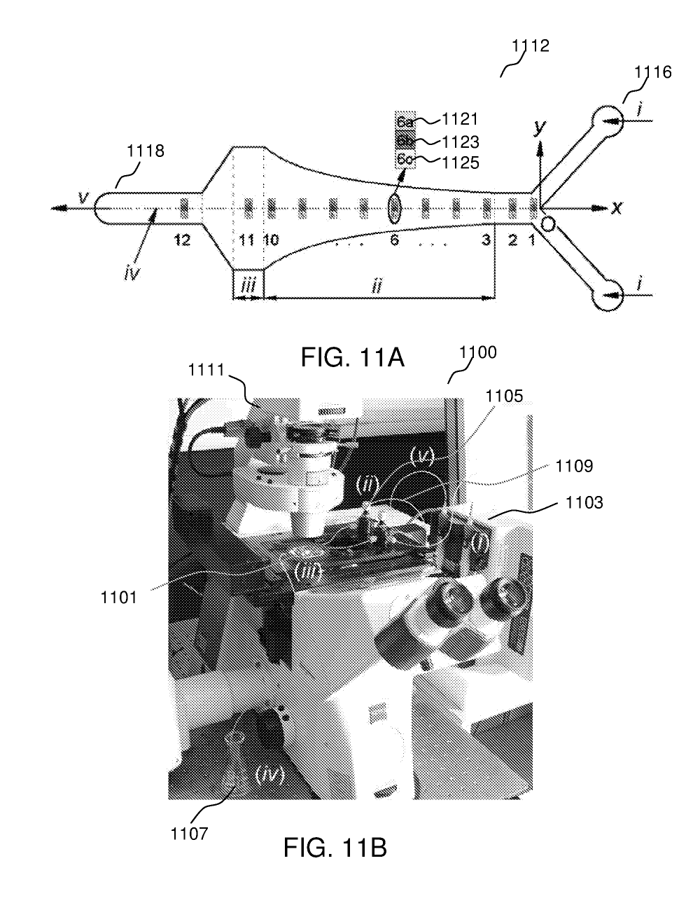

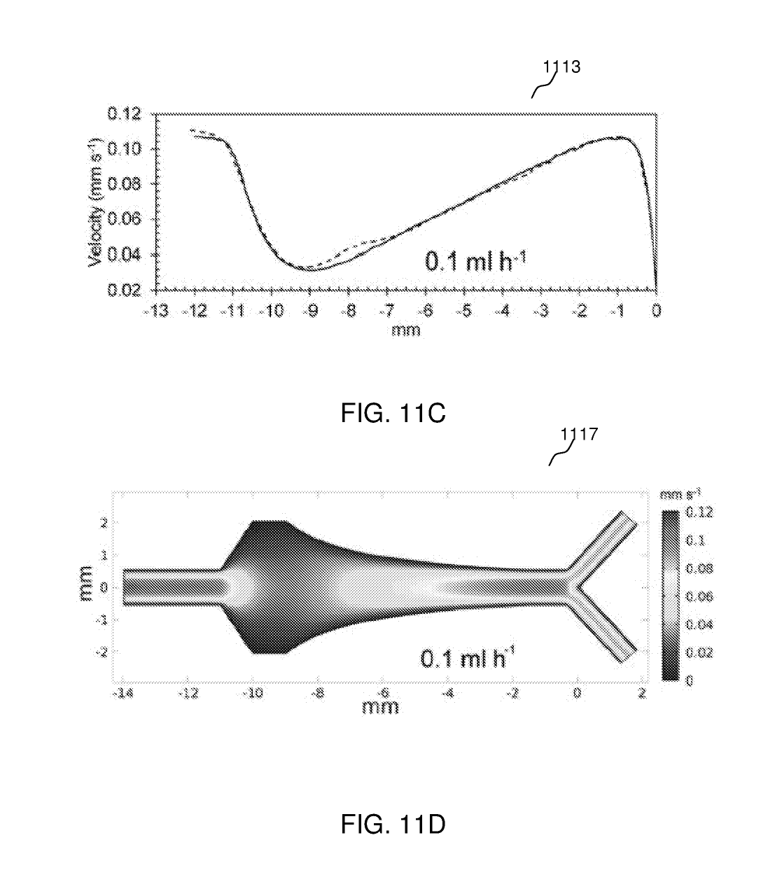

FIG. 11B shows a set-up of flow cell system 1100 on a confocal microscope. The flow cell system 1100 includes (i) syringe pump(s) 1103 to precisely regulate flow of media into the flow cell; (ii) valves 1105 for flexible media control; (iii) a newly-developed hyperbolic flow cell 1101 with a removable coverslip; (iv) effluent collectors 1107; and (v) tubing 1109 to connect different components of the system.

The flow cell used is in accordance with the various embodiments of the flow cell apparatus 100, 500, 600 as described herein. For this investigation, the flow cell was composed of one disposable acrylic (poly(methyl methacrylate)) plate carrying a channel 1112 (as shown in FIG. 11A) with a hyperbolic expansion, covered with a removable 22 mm×22 mm×0.17 mm microscopy glass coverslip.

FIG. 11A shows a top view of the channel 1112 with a hyperbolic channel profile. The channel 1112 has two inlets (i) 1116 feeding into a hyperbolic expansion leading to one outlet (v) 1118. For consistency of presentation, the flow direction is always indicated from right to left. The intersection of the two inlets was chosen as the reference zero point (O). 36 positions were selected for imaging, including 12 positions along the x direction numbered from 1 to 12, and three locations along the y direction for each position along x (iv is the flow cell centerline), numbered and colour coded as ‘a’ (top, cyan color 1121), ‘b’ (middle, magenta color 1123) and ‘c’ (bottom, yellow color 1125). An imaging window of 212.55 μm×212.55 μm at each position may be captured by using a 40×-magnification objective lens. Positions 3 to 10 are within the hyperbolic expansion (ii). Position 11 is at the exit of the expansion in the lowest flow rate region in the flow cell (iii). The channel depth was 0.98 mm for all experiments and can be easily modified for specific usage. Polymeric components of the flow cell, such as the acrylic plate carrying the channel profile, are designed to be disposable and can be mass produced by injection molding; therefore lowering its cost, which will aid in the wide adoption of this system.

A two-syringe infusion pump was employed to simultaneously control the media flow entering the two inlets 1116 of the flow cell. Three-way microvalves 1105 were used to provide flexible control of flow during various steps of the experimental process, such as removal of air bubbles arising from changing/replenishing media infused into the flow cell. PTFE Tubing 1109 was used for its desirable properties such as high temperature resistance, chemical inertness, low gas permeability and low friction coefficient.

The compact set-up of the flow cell system 1100 on a confocal microscope Zeiss LSM 780 (Carl Zeiss, Germany) for live imaging of biofilm development is shown in FIG. 11B. The assembly of the flow cell system 1100 is described in the following.

To set up the flow cell system 1100, glass coverslips on which biofilms developed, other components of the flow cell 1101, valves 1105 and tubes 1109 were first soaked in ethanol 70% v/v in DDW for 15 minutes. The components were then dried by using an air gun. Furthermore, potential biological contaminants on the cleaned coverslips and acrylic plates carrying the channel (all were disposable in each experiment) were removed by exposure to UV for 30 minutes. As further precaution, the flow cell 1101 was first assembled inside a bio safety cabinet. The list of components and assembly of the flow cell 1101 were in accordance with the various embodiments of the flow cell apparatus 100, 500, 600 as described herein. In contrast to most existing flow cells, the flow cell 1101 was specifically designed to have a removable coverslip fixed to the flow cell without the need of permanent bonding (for example adhesive or thermal bonding). Subsequently, the two three-way valves 1105, the syringe pump 1103 and effluent containers 1107 were connected to the assembled flow cell 1101. The system was then mounted onto a microscope 1111.

The simulation and validation of flow field will be described in the following.

A low flow rate (Q=0.1 ml h⁻¹ per inlet) was employed in this study. The channel geometry was constructed using SolidWorks (Dassault Systèmes SolidWorks Corp., MA, USA). The flow field was simulated using COMSOL Multiphysics 4.2a-Laminar Flow module (COMSOL Inc., MA, USA). M9 minimal growth medium supplemented with cassamino acids as shown in Table 1 below was used for both the simulation of the flow field and biofilm development experiments.

TABLE 1
Formula of M9 medium supplemented with casamino acids.

| | | | | | | Mass at |
|---|---|---|---|---|---|---|
| | | | | | | working |
| | | | | | Working | concen- |
| | Chemical | Chemical | | M_{w} | concen- | tration |
| No | name | formula | Brand | (g/mol) | tration | (g/L) |
| 1 | Calcium | CaCl₂•2H₂O | Merck | 147.01 | 0.1 mM | 0.01 |
| | chloride | | | | | |
| | dihydrate | | | | | |
| 2 | Magnesium | MgSO₄•7H₂O | Merck | 246.48 | 2.0 mM | 0.49 |
| | sulfate | | | | | |
| | heptahydrate | | | | | |
| 3 | M9 salt | | | | | |
| (a) | Sodium | Na₂HPO₄ | Fisher | 177.99 | 48.0 mM | 8.54 |
| | phosphate | | | | | |
| | dibasic | | | | | |
| (b) | Potassium | KH₂PO₄ | Merck | 136.09 | 22.0 mM | 2.99 |
| | dihydrogen | | | | | |
| | phosphate | | | | | |
| (c) | Sodium | NaCl | Merck | 58.44 | 9.0 mM | 0.53 |
| | chloride | | | | | |
| (d) | Ammonium | NH₄C1 | Merck | 53.49 | 19.0 mM | 1.02 |
| | chloride | | | | | |
| 4 | Glucose | D(+)− | BDH | 180.16 | 0.04% | 0.40 |
| | | Glucose | | | w/v | |
| 5 | Casamino | — | BD | — | 0.2% | 2.00 |
| | acids | | Bacto | | w/v | |

The density and viscosity of the M9 medium required for the simulation were measured to be 1016±2 kg m⁻³ and 1.09±0.01 mPa s respectively. Identical flow rates of 0.1 ml h⁻¹ were set at the two inlets and the single outlet was set at atmospheric pressure. No-slip boundary conditions (velocity=0) were imposed on the walls of the channel. The channel was meshed with physics-controlled mesh (calibrated for fluid dynamics) with maximum and minimum element sizes of 300 μm and 15 μm respectively. The simulated mid-plane velocity fields (velocity field at half-depth of the channel) and centerline velocities were plotted and subsequently validated experimentally by particle image velocimetry (PIV).

A 20% w/w glycerol in water solution was seeded with fluorescent polystyrene microspheres (3.2 μm diameter) at microsphere concentration of 0.1% w/w. Using a syringe pump, the fluid was infused into the flow cell at Q=0.1 ml h⁻¹ per inlet. The motion of the microspheres in the channel was captured using a high speed camera (Photron FASTCAM SA-5, Japan) on an inverted epi-fluorescence microscope (Nikon Ti-eclipse with Nikon Intensilight light source and Nikon TRITC filter cube, Japan) at magnification of 10× (Nikon Plan-Fluor 10× objective lens with N.A. 0.30, Japan). The mid-plane velocity was measured by positioning the focal plane of the objective lens at half-depth of the channel. The field of view of the high speed camera was 2048 μm×752 μm and the flow velocity along the central region of the flow cell (from x=0 mm to x=−12 mm) was sequentially measured over 9 imaging positions. Each imaging position was measured for 120 s and then was offset by 1250 μm along the x direction, resulting in an overlapping region of 798 μm wide between two consecutive positions.

Image acquisition parameters used for PIV can be seen in Table 3 below. The flow velocities were computed from the acquired images using a PIV program which was adapted from OpenPIV. The time-averaged velocities at each position were calculated and the flow velocity along the centerline of the flow cell was interpolated from the 9 positions. The measured velocities were then compared with the simulated velocity as shown in graph 1113 in FIG. 11C.

FIG. 11C shows a graph 1113 illustrating the comparison between simulated (continuous line) and measured (dashed line) flow velocity at centerline at flow rate Q=0.1 ml h⁻¹ per inlet. The horizontal axis is the x coordinates of the flow cell with the origin at O. From the graph 1113, the flow velocity decreased linearly from x=−1.49 mm to x=−8.99 mm resulting in a three times difference in magnitude at these two locations. FIG. 11D shows a schematic diagram 1117 of simulated mid-plane flow field at flow rate Q=0.1 ml h⁻¹ per inlet.

Biological experimental procedure and confocal imaging will be described in the following.

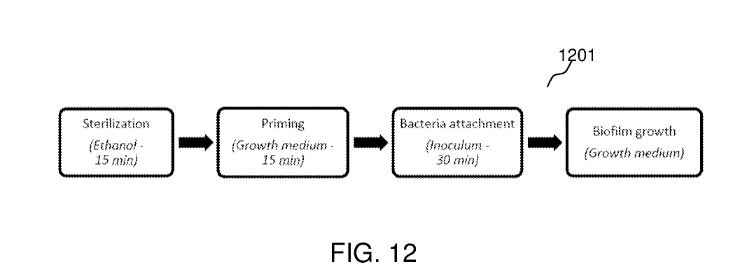

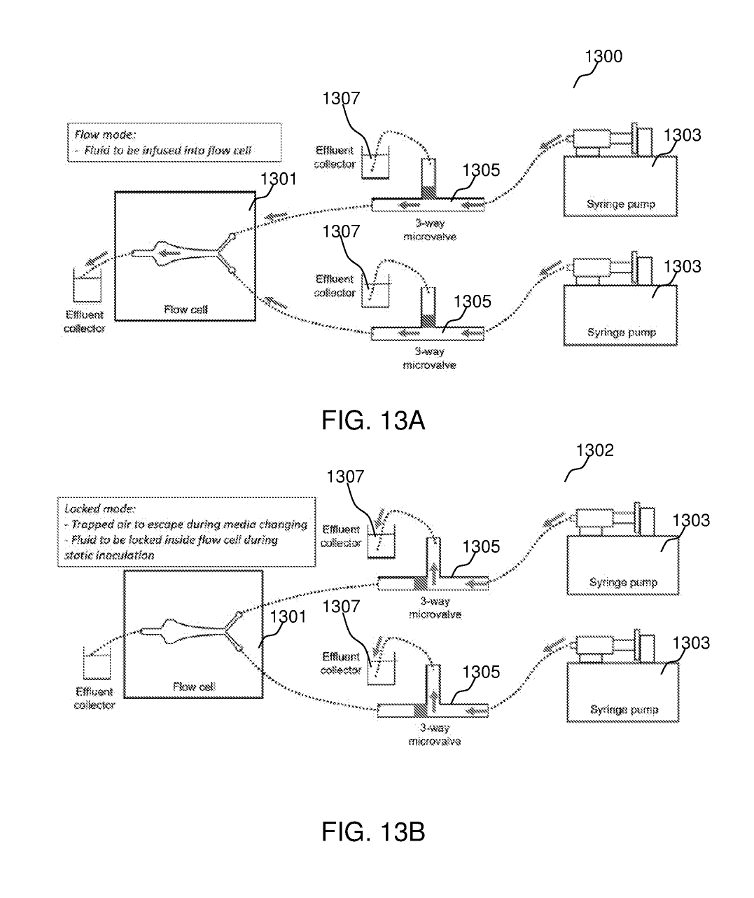

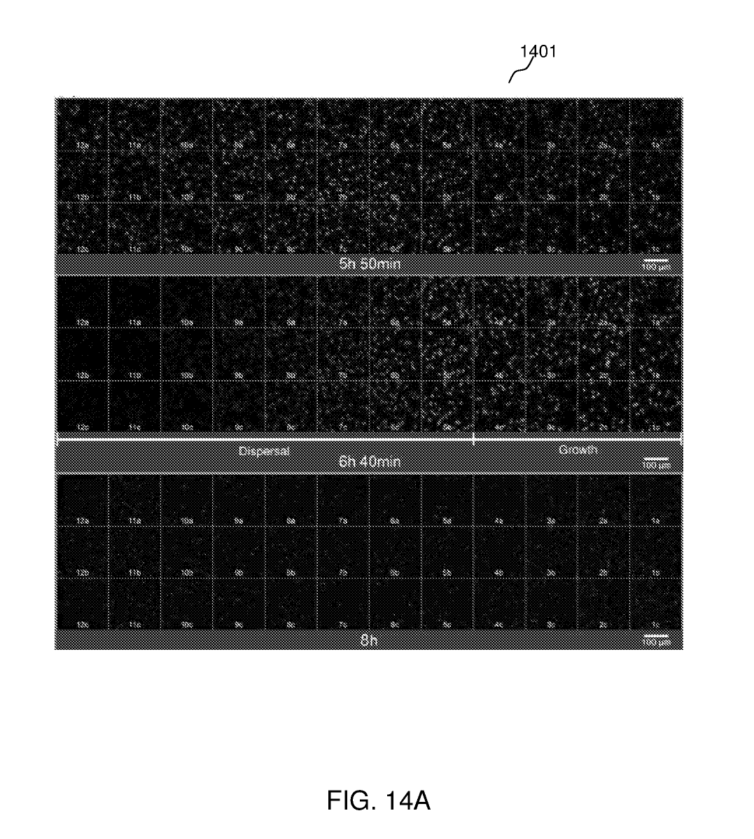

FIG. 12 shows a flow diagram 1201 of the experimental procedure of biofilm growth experiment to minimize risk of contamination according to various embodiments. FIGS. 13A and 13B show schematic diagrams of the flow cell system set-up 1300, 1302 in two different modes of operation respectively. The flow cell system 1300, 1302 are shown to be set-up with the aid of syringe pumps 1303 for precise control of fluid infused and three-way microvalves 1305 for flexible flow control that enables the switching of various types of media without air trapped inside the flow cell 1301.

FIG. 13A shows the schematic diagram 1300 of the flow cell system set-up in flow mode. In the flow mode, fluid from syringes mounted on syringe pumps 1303 is infused directly into the flow cell 1301 through the two inlets. This position is used during most of the time in an experiment except when the locked mode is in use.

FIG. 13B shows the schematic diagram 1302 of the flow cell system set-up in locked mode. In the locked mode, fluid with any trapped air inside due to changing of syringes is prevented from entering the flow cell 1301. Instead, the fluid, including any air trapped, is directed to the effluent collectors 1307. This position can be used during (1) Switching of media infused into the flow cell 1301 without air trapped. Media can be ethanol for priming, inoculum, growth media, etc. (2) Static inoculation—Inoculum is first infused into the flow cell 1301 by flow mode before switching the system to locked mode so that inoculum is locked inside the flow cell 1301 for cell attachment onto the coverslip surface.

In this case study, the flow mode was used as the default setting throughout the experimental procedure, except during media changing and during static inoculation where the locked mode was used.

At the start of the experiment, the flow cell was first sterilized with 70% v/v ethanol in DDW for 15 min at a flow rate of 1.0 ml h⁻¹ per inlet followed by priming the chamber with M9 medium for 15 min at the same flow rate to remove the ethanol. Next, the flow cell was inoculated with a suspension of defined strain of P. putida OUS82::GFP that expresses GFP constitutively. This strain was grown overnight in M9 medium and diluted to an optical density of 0.005 at 600 nm. The bacteria suspension at this concentration were measured to have 3.43×10⁶±5.51×10⁵ CFU ml⁻¹ counts. The inoculum was infused at an initial flow rate of 4 ml h⁻¹ per inlet for 1 min to completely fill the tubing from the valves to the two inlets. The flow rate was then reduced to 1 ml h⁻¹ per inlet for 4 min. Subsequently, the valves were switched to the locked mode for static inoculation. This event was defined as the reference zero time point t=0. Two syringes filled up with M9 media were precisely positioned onto the syringe pump. Any air trapped in the tubing was removed into the two effluent collectors, for example as shown in FIG. 13B. At t=30 min, M9 media was infused at Q=0.1 ml h⁻¹ and the valves were switched back to the flow mode to allow M9 media flow into the flow cell. This event marked the onset of biofilm development experiment. The experiment was conducted at 24° C.

Imaging of the flow cell was conducted on a confocal microscope Zeiss LSM 780 with a 40× objective (Plan-Apochromat, N.A. 0.95, Korr). This microscope is equipped with a motorized stage (1300×100 DC, Carl Zeiss, Germany). 36 positions were carefully selected along the chamber for real-time imaging. These defined positions were accurately recorded in the microscope software (Zen 2011) and the intersection of the two inlets was defined as the zero reference point in the x-y plane (e.g., as shown in FIG. 11A). Z-stacks of the biofilm were acquired by using 488 nm excitation wavelength and the GFP emission was detected at 493-598 nm. Z-stacks of 12 slices at 0.78 μm intervals were used. Collection of all images at the 36 positions in an imaging cycle was carried out over a duration of 7 min. Imaging cycles were initiated every 10 min over the duration of 8 h 20 min.

Quantification of three-dimensional biofilm confocal images will be described in the following.

To quantify biofilm development, three-dimensional stacked images were used and biofilm bio-volume parameters were obtained by computation using Imaris (Bitplane, Switzerland). Additionally biofilm clustering was calculated throughout the duration of the experiment at 36 positions. The bio-volume of individual biofilm clusters i at position p at imaging cycle n, defined as Vₚₙᵢ, were calculated using the surface segmentation algorithm of Imaris. The segmentation parameters used for the above computation were defined as: (a) absolute intensity threshold of 10; and (b) minimum object size of 3 voxels (each voxel is a cuboid of 0.42 μm×0.42 μm×0.78 μm). The computed bio-volume is defined to be the volume of the bacteria cells excluding the additional volume of the extra-cellular polymeric matrix. The same segmentation parameters were applied in all positions and cycles.

Vₚₙ—total bio-volume per imaging window at position p and imaging cycle n, was calculated by summing the bio-volumes of all the clusters in the imaging window of 212.55 μm×212.55 μm determined by the image acquisition parameters and the specific objectives used.

$${V}_{pn} = {\sum }_{i = 1}^{{N}_{pn}} {V}_{pni} \qquad ( 1 )$$

where p is the position (p=1 to 36), n the imaging cycle number (n=1 to 47). The conversion between imaging cycle n and actual growth time t is shown in Table 2 below.

Nₚₙ is the total number of clusters in the imaging window at position p and imaging cycle n. Nₚₙ is not constant but varied at different positions and different imaging cycles.

Vₚₙ was normalized against the total bio-volume at the respective position using a reference time point at 2 h (imaging cycle 10—Vₚ₁₀) in the experiment to allow for comparison between different positions. Time point 2 h was chosen because the bacteria would have permanently attached to the surface by that time. The normalized total bio-volume at position p and imaging cycle n (Vnormₚₙ) is calculated as follows:

$${Vnorm}_{pn} = \frac{{V}_{pn}}{{V}_{p 10}} \qquad ( 2 )$$

The apparent growth rate at position p was assumed to follow an exponential equation described as:

Vₚₙ(t)=Vₚₙ₀e^{g}^{ₚ}^{t} (3)

where Vₚₙ(t) is the total bio-volume at position p at time t (i.e. imaging cycle n), Vₚₙ₀ is the initial total bio-volume at position p (i.e. at imaging cycle n₀), gₚ is the apparent growth rate at position p and has positive value, t the time. The average growth rate at position p (g̅ₚ̅) was calculated by fitting Vₚₙ during the period from the start of experiment (n₀=1) to the point of maximal observed growth (time at which total bio-volume reached its maximal value).

The apparent biofilm removal rate (including dispersal and sloughing) at position p was assumed to follow an exponential equation:

Vₚₙ(t)=Vₚₙ₀e^{r}^{ₚ}^{t} (4)

where rₚ is the apparent biofilm removal rate at position p and has negative value. rₚ was fitted from the start of dispersal (i.e. the cycle immediately after the maximal growth at each position) to the end of experiment.

The doubling time at position p, t_{dp}, was calculated by:

$${t}_{{d}_{p}} = \frac{ln 2}{{g}_{p}} \qquad ( 5 )$$

The distribution of cluster sizes at position p, at imaging cycle n, was plotted by sorting Vₚₙᵢ in ascending order against the total number of clusters at that position Nₚₙ (with p being the position, p=1 to 36).

The cluster size and its spatial distribution at each defined time point is represented by the bubble plot. Each bubble represents an individual cluster i at position p taken in imaging cycle n. Its diameter, Dₚₙᵢ, was computed from Vₚₙᵢ by assuming the cluster as a sphere as follows:

$${D}_{pni} = \sqrt[3]{\frac{6 {V}_{pni}}{π}} \qquad ( 6 )$$

The centre of the bubble was taken as the centroid of the respective clusters.

A summary of the experimental parameters and specifications of image acquisition is presented in Table 3 below.

TABLE 3
Summary of experimental parameters and specification
PIV measurement

| Microscope | Nikon Ti-eclipse epi-fluorescence | |
|---|---|---|
| | inverted microscope | |
| Objective | Plan-Fluor 10x N.A. 0.30 | |
| Camera | Photron FASTCAM SA-5 | |
| Scaling (per Pixel) | 2 μm × 2 μm | |
| Image Size (Pixels) | 1024 × 376 | |
| Image Size (scaled) | 2048 μm × 752 μm | |
| Time between image pair | 400 | ms |
| Exposure time | 25 | ms |
| Image pair acquisition frequency | 2.5 | Hz |
| Number of positions | 9 | |
| Measurement duration for one | 120 | s |
| position | | |

Confocal imaging

| Equipment | Zeiss LSM 780, AxioObserver | |
|---|---|---|
| Objective | Plan-Apochromat 40x N.A. 0.95 Korr | |
| Detection wavelength | 493-598 | nm |
| Scaling (per Pixel) | 0.42 μm × 0.42 μm × 0.78 μm | |
| Image size (Pixels) | 512 × 512 | |
| Image size (scaled) | 212.55 μm × 212.55 μm | |
| Pixel time | 1.27 | μs |
| Frame time | 0.78 | s |
| Interval between two cycles | 10 | min |
| Number of positions for each cycle | 36 | positions |
| Z-stack at each position | 12 slices (8.618 μm) | |
| Total imaging period | 8 h 20 min | |
| Total number of cycles | 47 | cycles |
| Total number of images | 20,304 | |
| Size of data set | 9.94 | GB |

Results of the study will be described in the following.

The chamber used has a channel 1112 with two inlets 1116 feeding into a hyperbolic expansion that then leads to one outlet 1118 (as shown in FIG. 11A). The hyperbolic expansion generates a linearly decreasing shear rate along the channel centerline (zone ii in FIG. 11A). The flow field (as shown by graph 1113 in FIG. 11C and schematic diagram 1117 in FIG. 11D) was first simulated and then experimentally validated by PIV, with good agreement between the simulated and measured flow velocities (as shown in graphs 1113 in FIG. 11C).

Real-time high-content 3D imaging will be described in the following.

The biofilm development of GFP tagged P. putida under defined flow fields was observed using confocal laser scanning microscopy at real-time, high-content, and high resolution. The experiment which was conducted for up to 8 h 20 min generated up to 20,304 images, taken at multiple locations with an interval of 10 min.

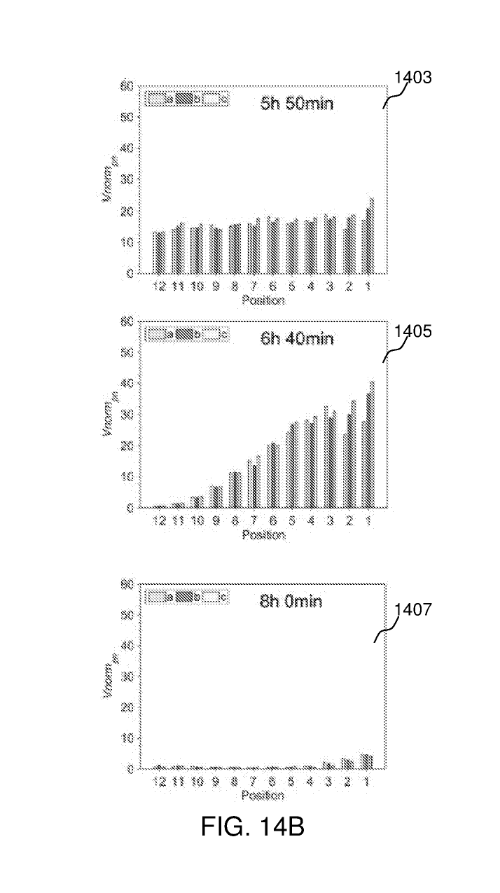

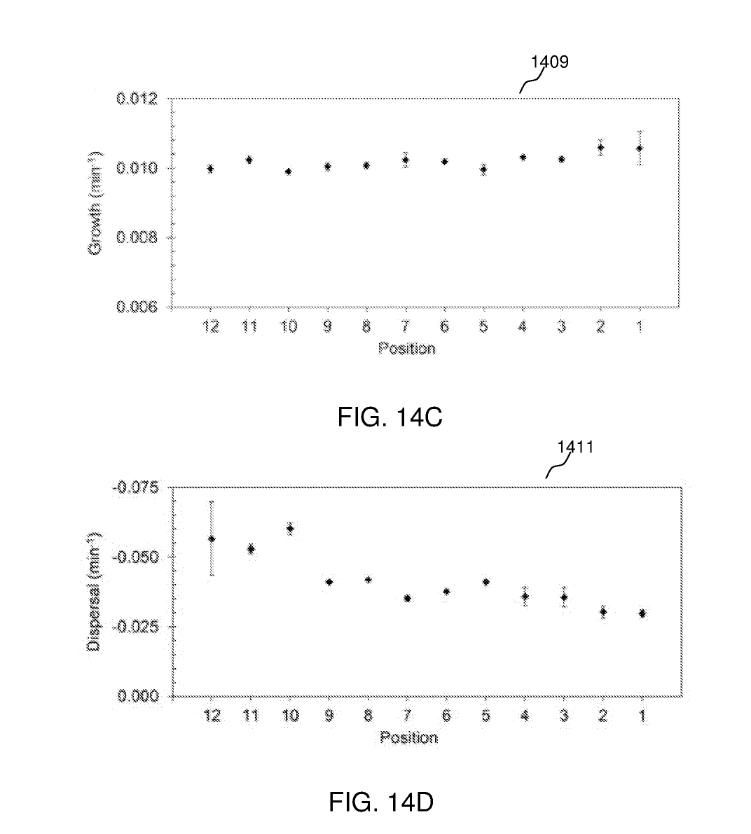

FIGS. 14A to 14D show experiment data illustrating the dynamic nature of P. putida OUS82::GFP biofilm formation and dispersal at flow rate Q=0.1 ml h⁻¹ per inlet.

FIG. 14A shows confocal microscopy images 1401 of P. putida biofilm formation and dispersal at flow rate Q=0.1 ml h⁻¹ per inlet at 36 positions at selected significant time points. The image at each time point is a collage of 36 images, each of which is the maximum intensity projection from the Z-stack of the respective location. (Top) At 5 h 50 min, the microcolonies at downstream positions 12a-c (i.e. leftmost images) reached their maximal growth. A few bacteria started swimming around and began leaving the microcolonies. This was the start of dispersal at these locations. Growth continued at positions 1-11 during this time. Dispersal then further propagated upstream over the subsequent two hours. (Middle) At 6 h 40 min, dispersal occurred at positions 12-5 while the microcolonies at positions 4-1 continued growing. (Bottom) At 8 h, dispersal was evident at all positions. Most cells were flushed downstream towards the outlet of the flow cell.

The same growth and dispersal behaviours were observed with repeated experiments at similar temporal resolution of ±10 min.

Quantification of the dynamics of the biofilm behaviour was achieved by analysing the large number of collected confocal microscopy images.

FIG. 14B shows graphs 1403, 1405, 1407 representing the normalized total bio-volume per imaging window, Vnormₚₙ, of the biofilm shown in FIG. 14A at selected significant time points. In the top graph 1403, at 5 h 50 min, microcolonies at positions 12 a-c reached their highest bio-volume corresponding to their maximal growth. There are small variations in Vnormₚₙ at the three positions along y direction for each position along x axis (apart from locations 1 and 2), showing that biofilm growth is consistent. In the middle graph 1405, the gradually increasing trend of Vnormₚₙ from positions 12 to 1 shows the different stages of microcolony development along the flow direction at 6 h 40 min. Vnormₚₙ at positions 12-5 are decreasing indicating dispersal while those at positions 4-1 are increasing indicating continued growth. In the bottom graph 1407, at 8 h 0 min, Vnormₚₙ at all positions drop significantly showing that the biofilm had dispersed and bacteria were flushed out of the flow cell. All data may be normalized against the total bio-volume at the respective positions at 2-h time point.

The apparent growth rate (FIG. 14C) and apparent biofilm removal rate (FIG. 14D) at the same position, were computed by fitting Vnormₚₙ to equations 3 and 4 respectively. The average apparent growth rate at positions 1-12 is shown in a graph 1409 in FIG. 14C. Each point is the average of gₚ at the resp̅e̅ctive ‘a’, ‘b’, ‘c’ positions. A graph 1411 is shown in FIG. 14D illustrating the average apparent biofilm removal rate at positions 1-12. Each point was obtained by averaging the calculated rₚ at the respective ‘a’, ‘b’, ‘c’ positions.

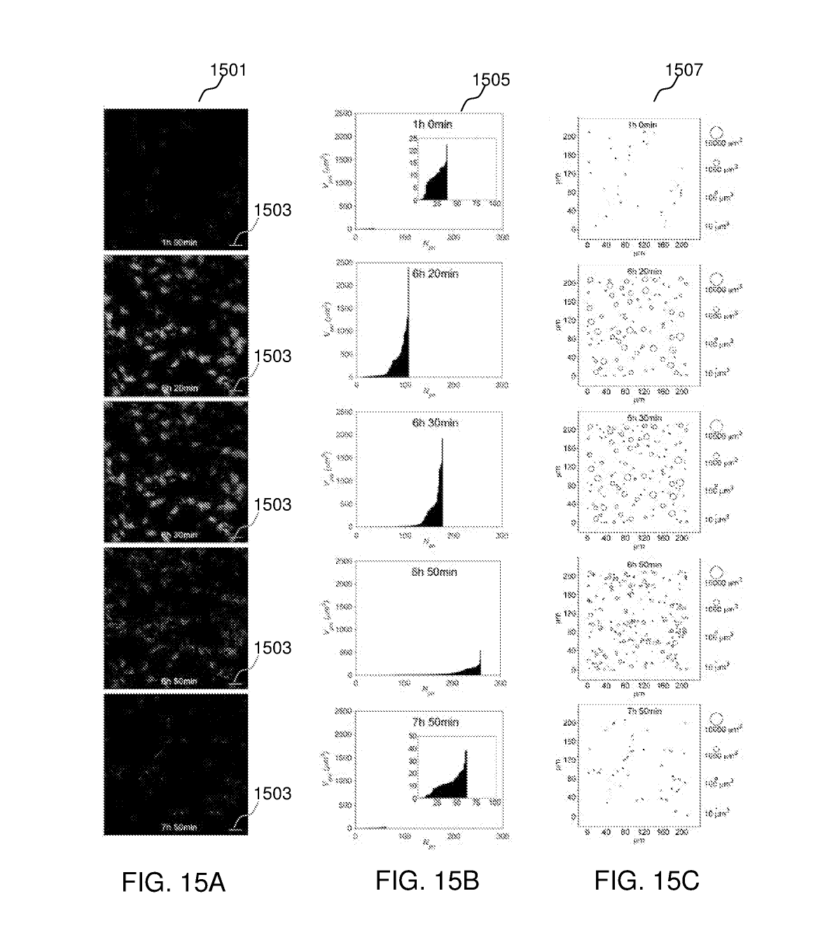

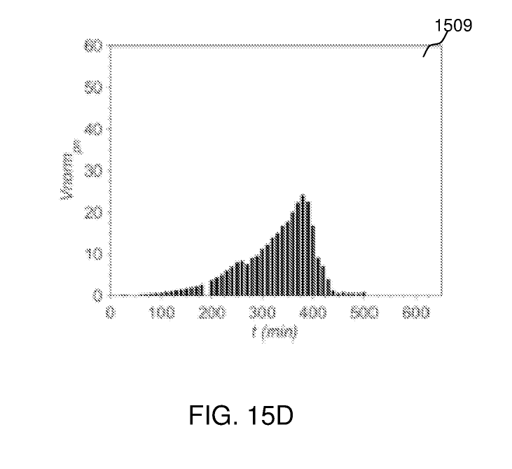

FIGS. 15A to 15D show experimental data illustrating the dynamics of P. putida OUS82::GFP biofilm development (formation and dispersal) at position 7a at significant time-points under flow rate Q=0.1 ml h⁻¹ per inlet.

FIG. 15A shows confocal images 1501 over the time course of initial biofilm development—from attachment to dispersal: 1 h (initial attached bacteria), 6 h 20 min (maximal growth of biofilm cluster before start of dispersal), 6 h 30 min (commencement of dispersal), 6 h 50 min (dispersal of biofilm) and 7 h 50 min (fully dispersed biofilm, some P. putida cells that remained on the surface resembled filaments. The confocal images are taken at magnification 40×. In the confocal images 1501, the scale bar 1503 is 20 μm.

The distribution of cluster size at position 7a corresponding to the biofilm images in FIG. 15A is illustrated in graphs 1505 in FIG. 15B. The y-axis is the individual cluster bio-volume, Vₚₙᵢ, while the x-axis is the total number of clusters present in the imaging window, Nₚₙ. The increased in height of the distribution indicates growth while the spreading of the distribution to the right indicates dispersal. As the biofilm clusters increased in size, the distribution shows an increasing height (1 h to 6 h 20 min). At the onset of dispersal, the cluster size distribution expands abruptly to the right (6 h 20 min to 6 h 30 min). The distribution continues expanding towards the right while its height gradually decreases (6 h 30 min to 7 h 50 min) during dispersal, indicating bacteria were leaving the microcolonies. The distribution moves to the left with significant reduction in the distribution height when most bacteria were flushed out of the flow cell (7 h 50 min).

One hour into the experiment, the attached bacteria were mostly arranged as single cells and small clusters have bio-volumes of less than 25 μm³. At t=6 h 20 min, the biofilm reached the maximal growth. At this time, the largest cluster has a bio-volume of 2388 μm³ (the highest peak in the distribution FIG. 15B). The shift of the distribution to the right soon afterwards indicates a surge in the total number of clusters, and the drop in the clusters bio-volume represents the commencement of dispersal. During the next 30 min, the cluster size distribution shifts further to the right. This corresponds to an additional increase in the number of smaller clusters as a result of clusters breaking up.

FIG. 15C shows bubble plot 1507 of the spatial distribution of Vₚₙᵢ for the corresponding time point in FIG. 15A. A circle represents a cluster; its area represents the magnitude of Vₚₙᵢ. The centers of the circles are taken as the centroid of the respective clusters. The bubble plot (FIG. 15C) illustrates the development of single attached bacteria into clusters and for nearby clusters merged to form a larger cluster. The bubbles initially increase in diameter and eventually break into smaller components during dispersal. This representation of bio-volume data can further be used for modelling and spatial pattern recognition to gain better insights.

FIG. 15D shows a graph 1509 illustrating the normalized total bio-volume, Vnormₚₙ, at Position 7a over time, t. From the normalized total bio-volume, Vnormₚₙ (as shown in FIG. 15D), the time at maximal growth and commencement of dispersal is identified. The time of maximal growth is defined as the exact time at which Vnormₚₙ is maximal. The time at the start of dispersal is identified from the decrease in Vnormₚₙ, expressed immediately after the highest peak of Vnormₚₙ (see FIG. 15D).

By fitting Vnormₚₙ to equations 3 and 4, the apparent growth rate (FIG. 14C) and apparent biofilm removal rate (FIG. 14D) at the same position were computed respectively.

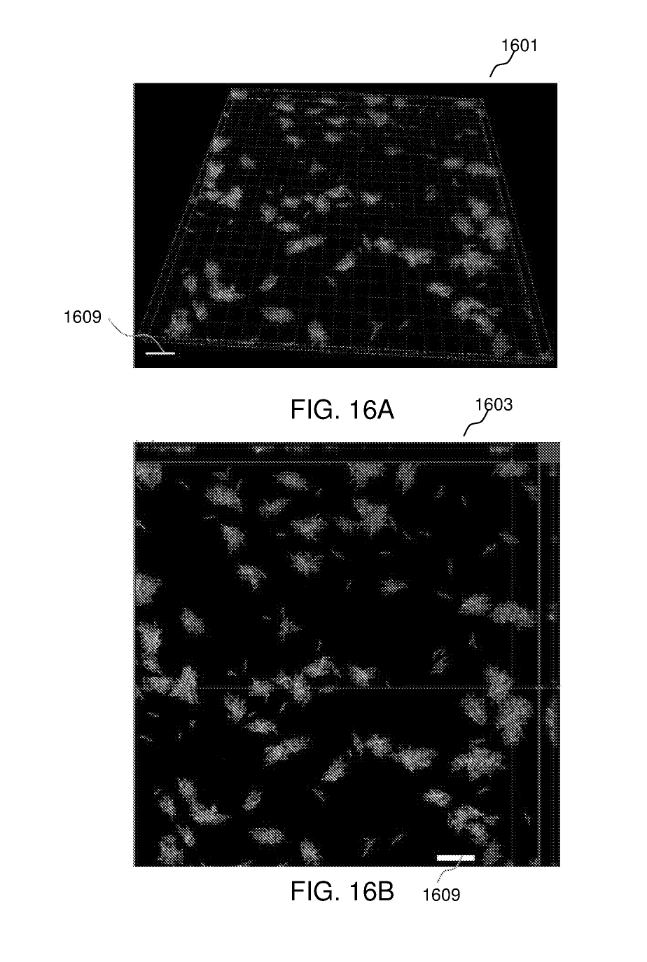

FIGS. 16A and 16B show microcolonies structure of P. putida OUS82::GFP model biofilm developed at position 7a. FIGS. 16A and 16B show a three-dimensional view 1601 and a cross-section view 1603 of the confocal image of FIG. 15A at 6 h 20 min time point respectively. FIG. 16A shows a three-dimensional confocal image 1601, constructed from a Z-stack of 12 slices at 0.78 μm interval, of biofilm at 6 h 20 min under low flow rate Q=0.1 ml h⁻¹ per inlet. FIG. 16B shows a cross-section view of microcolonies that reached their maximal growth after 6 h 20 min under low flow rate Q=0.1 ml h⁻¹ per inlet. In FIGS. 16A and 16B, the scale bars 1609 are 20 μm. In the cross-section views in FIG. 16B, the top and side images represent x-z and y-z planes respectively.

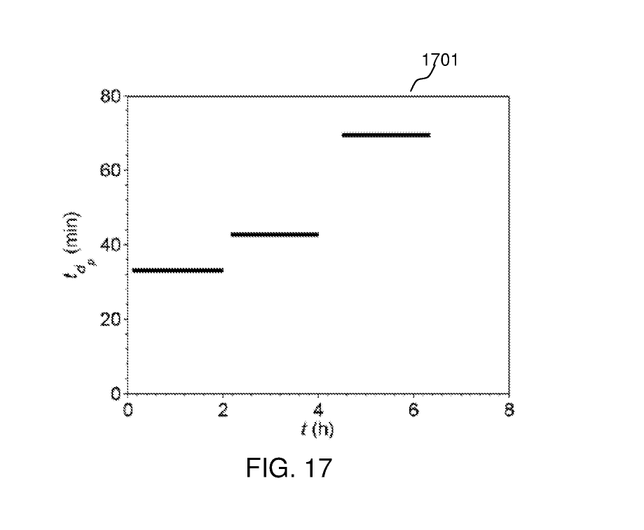

FIG. 17 shows graph 1701 illustrating the doubling time of biofilm, t_{dp}, at position 7a over defined periods of the experiment (t=0 h 6 min-2 h 0 min, 2 h 10 min-4 h 0 min, and 4 h 30 min-6 h 20 min). The apparent growth rate, gₚ, was fitted over defined periods. By using equation 5, t_{dp} over these defined periods was calculated. It was assumed that gₚ, and hence t_{dp}, is constant over each of the defined periods. All the fittings have R² higher than 0.98. From the graph 1701 in FIG. 17, the doubling time increased significantly from 43 min at 2 h to 69 min at 6 h 20 min when the biofilm was exposed to low flow rate of Q=0.1 ml h⁻¹ per inlet.

This experimental approach that merges microbial ecology and engineering provides a high precision platform to create reproducible well-defined conditions for biofilm growth, while enabling long-term, high-content, real-time imaging of dynamic biofilm development at high spatio-temporal resolution.

The total volume of the growth chamber is small (35 μl). Thus, the required volume of operating liquid (i.e. medium, reagents, stains, etc.) is small. Therefore, a wide range of shear rate can be accurately generated in the laboratory. Our compact flow cell system facilitates the implementation of experimental set-up, especially for fast image acquisition high-resolution microscopy.

The protocol for the operation of the flow cell is robust. It also allows for dynamic observations of biofilm development and its correlation to well-controlled environments. Then, we can conduct analysis on key parameters of biofilm development behavior: bio-volume, cluster distribution, biofilm growth and removal rates as well as doubling time.

The dynamic nature of biofilm formation and dispersal of P. putida, seen for the first time, can be revealed only when observing it under high resolution of single micrometer and at ten minutes interval.

By merging of engineering and microbial ecology, various embodiments have provided a rigorous methodology to quantify biofilm development at high resolution using our newly-developed flow cell and robust protocol. Various embodiments have also provided a high precision flow cell to create well-defined and reproducible microenvironments. This may enable high-content confocal laser scanning microscopic observation and quantification using the model biofilm organism Pseudomonas putida.

Using this high resolution approach, it was observed, for the first time, unpredicted dynamics of P. putida biofilm formations followed by total dispersal that are closely correlated to the microenvironments. These biofilm behaviour phenomena were highly reproducible, despite the heterogeneous nature of biofilm.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A flow cell apparatus comprising:
a channel plate having
a channel recessed into a surface of the channel plate, and
a groove recessed into the surface of the channel plate, the groove configured to surround the channel;
a seal shaped and receivable in the groove;
a substrate;
a backing plate; and
a fastening element configured to removably attach the channel plate to the backing plate with the substrate sandwiched between the channel plate and the backing plate to bear the seal against the channel plate with the substrate.

2-5. (canceled)

6. The flow cell apparatus as claimed in claim 1, wherein the channel comprises an expanded region followed by a contracted region, wherein the expanded region further comprises a hyperbolic expanded region.

7. (canceled)

8. The flow cell apparatus as claimed in claim 1, wherein the channel comprises a contracted channel, wherein the contracted channel further comprises a hyperbolic contacted channel.

9. (canceled)

10. The flow cell apparatus as claimed in claim 1, wherein the backing plate comprises a window, and wherein an edge of the window is tapered.

11-12. (canceled)

13. The flow cell apparatus as claimed in claim 1, wherein the backing plate comprises a winged portion.

14-17. (canceled)

18. The flow cell apparatus as claimed in claim 1, wherein the fastening element is configured to self-lock.

19. The flow cell apparatus as claimed in claim 1, wherein the fastening element comprises a quick-release fastening element.

20. The flow cell apparatus as claimed in claim 1, wherein the fastening element comprises a snap-fit fastener and a compression spring.

21. (canceled)

22. The flow cell apparatus as claimed in claim 1, wherein the fastening element comprises a clamp.

23. The flow cell apparatus as claimed in claim 1, wherein at least one of the substrate or the channel plate comprises one or more markers.

24. (canceled)

25. The flow cell apparatus as claimed in claim 1, wherein the channel plate comprises two or more channels recessed into the surface of the channel plate.

26. (canceled)

27. The flow cell apparatus as claimed in claim 25, wherein the channel plate comprises two or more grooves, and wherein each of the two or more grooves is configured to surround a corresponding channel of the two or more channels along the boundary of the corresponding channel.

28. The flow cell apparatus as claimed in claim 1, wherein the channel plate comprises two or more layers, and wherein a first layer of the two or more layers comprises a base channel recessed into a surface of the first layer, and wherein a second layer of the two or more layers comprises a channel-shaped-through-hole in the second layer, and wherein the first layer is configured to receive the second layer to join the base channel and the channel-shaped-through-hole to form the channel in the channel plate.

29. The flow cell apparatus as claimed in claim 28, wherein each of the base channel and the channel-shaped-through-hole comprises a channel profile different from each other.

30. The flow cell apparatus as claimed in claim 28, wherein the channel plate comprises two layers, and wherein the groove is recessed into a surface of the second layer and configured to surround the channel-shaped-through-hole along a boundary of the channel-shaped-through-hole.

31. (canceled)

32. A method of analyzing biofilm development, the method comprising quantifying biofilm development in a flow cell apparatus, wherein the flow cell apparatus comprises:
a channel plate having a channel recessed into a surface of the channel plate, and a groove recessed into the surface of the channel plate, wherein the groove is configured to surround the channel;
a seal shaped and receivable in the groove;
a substrate;
a backing plate; and
a fastening element configured to removably attach the channel plate to the backing plate with the substrate sandwiched between the channel plate and the backing plate to bear the seal against the channel plate with the substrate.

33. The method as claimed in claim 32, further comprising imaging biofilm development at multiple locations along the channel in the flow cell apparatus at a predetermined time interval.

34. (canceled)

35. The method as claimed in claim 32, further comprising determining biofilm cluster distribution from the quantified biofilm development.

36. The method as claimed in claim 32, further comprising determining at least one of biofilm growth rate, biofilm removal rate, or biofilm doubling time from the quantified biofilm development over a period of time.

37-38. (canceled)

39. A flow system comprising:
a flow cell apparatus including:
a channel plate having a channel recessed into a surface of the channel plate, and a groove recessed into the surface of the channel plate, wherein the groove is configured to surround the channel,
a seal shaped and receivable in the groove,
a substrate,
a backing plate, and
a fastening element configured to removably attach the channel plate to the backing plate with the substrate sandwiched between the channel plate and the backing plate to bear the seal against the channel plate with the substrate;
a valve connected to the channel of the flow cell apparatus; and
a collector connected to the valve,
wherein the valve is configured to direct a fluid flow through the valve into the channel of the flow cell apparatus in a flow mode, and further configured to direct the fluid flow through the valve into the collector and hold the fluid in the channel of the flow cell apparatus in a locked mode.

40. (canceled)
